(19) United States
(12) Patent Application Publication
(10) Pub. No.: US 2026/0224610 A1
(43) Pub. Date: Aug. 6, 2026

(54) A BIOACTIVE POLY(LACTIC-CO-GLYCOLIC ACID) (PLGA) MATERIAL, RELATED PRINTED STRUCTURE AND RELATED METHODS THEREOF

(71) Applicants: AGENCY FOR SCIENCE, TECHNOLOGY AND RESEARCH, Singapore (SG); SINGAPORE HEALTH SERVICES PTE LTD, Singapore (SG)

(72) Inventors: Mohammad Asyraf Bin ABBAS, Singapore (SG); Peili TEO, Singapore (SG); Yew Chin TEO, Singapore (SG); Eun Ju PARK, Singapore (SG); Jiayi GUO, Singapore (SG); Wan Loong James MOK, Singapore (SG)

(73) Assignees: AGENCY FOR SCIENCE, TECHNOLOGY AND RESEARCH, Singapore (SG); SINGAPORE HEALTH SERVICES PTE LTD, Singapore (SG)

(21) Appl. No.: 19/159,743

(22) PCT Filed: Feb. 27, 2024
(86) PCT No.: PCT/SG2024/050107

§ 371 Date: Aug. 26, 2025

(30) Foreign Application Priority Data
Feb. 27, 2023 (SG) 10202300519W

(51) Int. Cl. A61K 31/765; A61K 47/34; A61P 17/02; B29C 64/118; B33Y 70/00; B33Y 80/00

(52) U.S. Cl. CPC A61K 31/765; A61K 47/34; A61P 17/02; B29C 64/118; B33Y 70/00; B33Y 80/00

(57) ABSTRACT

There is provided a regenerative material for treatment of wounds, the material comprising: (i) a base poly(lactic-co-glycolic acid) (PLGA); (ii) a bioactive poly(lactic-co-glycolic acid) (PLGA) copolymer; and (iii) pores patterned on said material. Also provided are a medical device comprising said regenerative material, medical uses of said regenerative material, and a method of preparing said regenerative material.

### TECHNICAL FIELD

The present disclosure relates broadly to a bioactive poly(lactic-co-glycolic acid) (PLGA) material and related printed structure. The present disclosure also relates to a method of preparing said bioactive PLGA material, and related methods and uses.

### BACKGROUND

Effective wound care requires a multi-modal approach, including wound bed optimization, management of medical conditions, and consistent follow up. Importantly, wound care products play a key role in wound bed optimization.

Currently, there are many wound care products available commercially. Most of the available advanced dressings are intended to maintain a moist wound environment (e.g., alginates, foams, hydrocolloids and hydrogels) or to provide antimicrobial properties (e.g., silver-based dressings). However, these currently available dressings have several shortcomings. For example, silver-based dressings are cytotoxic and can lead to epidermis damage (e.g. Acticoat™, Smith & Nephew), while polyurethane-based foam dressings tend to cause maceration of peri-wound skin when they become saturated (e.g. Allevyn™, Smith & Nephew).

Recently, there are many significant biomedical innovations aimed at improving rates of healing by facilitating rapid revascularization, re-epithelialization, and healing of wound beds. These strategies include the application of active biologic, biomembrane or scaffold based wound dressings, that act as skin substitutes to expedite wound healing. Epidermal stem cells may also be utilized to impregnate biocompatible substrates to promote skin regeneration. However, these cellular products have several limitations and are far from desirable. For example, they tend to have short shelf lives, poor thermal stability and low ease of handling. These products also tend to be expensive, making it relatively inaccessible to the majority of patients.

As a result of disadvantages and drawbacks, most currently available wound care products are designed only for infection control or exudate management. Few possess skin regeneration properties, and even if they do, such products are often expensive and inaccessible to patients.

One of the most prevalent forms of wounds include burns, which are the third leading cause of preventable death in children worldwide. The most common types of burns are thermal burns from scalding or contact with hot surfaces such as irons, stoves etc. Most paediatric burns are minor but severe burns in children have higher mortality than non-elderly adults with similar burns. Currently, wound care of second degree partial thickness burns commonly involved covering with silver-based antimicrobial dressings after debridement and blister removal, but there is a risk that such dressings could possibly delay wound healing due to cytotoxicity of silver. Biological dressings such as Biobrane may be used for partial thickness wounds. However, such dressings (i.e. without seeding with stem cells prior to use) have been reported to be slow in healing. The use of stem cells is often required to facilitate healing. However, cell-based treatment (e.g., stem cell access) is extremely costly, can be challenging in some hospitals, and can involve multi-step process that limit their usage during emergency treatment.

The current gold standard in deep partial thickness and full thickness burn wounds is skin autograft. However, skin autograft has its limitations, e.g., when there is limited harvestable skin for grafting and where donor site scarring is unacceptable. In such situations, skin substitutes are used instead. Biobrane is a popular skin substitute used in burns treatment. However, as mentioned, for Biobrane to work effectively, it is required to be pre-seeded with fibroblasts before use. As cells may take 2-3 weeks to culture, such a time lag would constraint its use. Without cells, wound epithelisation can be slow and infection can occur, leading to complications. Biobrane is required to be used in sterile environment within 48 hours of injury and can only be used on wounds with low bacteria load. Biobrane is also extremely costly at about S$800-S$1,000 per sheet. Furthermore, there has been adverse reaction reported by the FDA regarding unknown infection from the use of Biobrane. Other options include Integra, a bovine collagen dermal substitute, but this bovine collagen dermal substitute has been reported to have a tendency to shrink over time, high infection complication and failure rate of up to 30%. Furthermore, the staged procedure involved in Integra use has led to the unpopularity of this scaffold. To date, finding a material that could be suitably used as a skin substitute for wound care remains a challenge.

In view of the above, there is a need to address or at least ameliorate the above-mentioned problems. In particular, there is a need to provide a material with skin and/or tissue regenerative properties for a cost efficient and yet effective treatment of wounds.

### SUMMARY

In one aspect, there is provided a regenerative material for treatment of wounds, the material comprising:
(i) a base poly(lactic-co-glycolic acid) (PLGA);
(ii) a bioactive poly(lactic-co-glycolic acid) (PLGA) copolymer; and
(iii) pores patterned on said material.

In one embodiment, each pore comprises a polygonal shape.

In one embodiment, each pore has a minimum circumscribed circle diameter of at least 100 μm.

In one embodiment, the distance between the pores is from 100 μm to 300 μm.

In one embodiment, the material comprises a buffer zone surrounding the pores.

In one embodiment, the material has an infill density of from 70% to 100%.

In one embodiment, the ratio of the (i) base PLGA to (ii) bioactive PLGA copolymer present in the material is from about 60.0-99.9:0.1-40.0.

In one embodiment, the material comprises from 60 wt % to 99.9 wt % of the base PLGA.

In one embodiment, the material comprises from 0.1 wt % to 40 wt % of the bioactive PLGA copolymer.

In one embodiment, the material comprises a three-dimensional (3D) 20 printed structure or part.

In one embodiment, the bioactive PLGA copolymer comprises a bioactive PLGA copolymer with a poly(norbornene-dicarboximide) backbone having one or more repeating units represented by general formula (I) and one or more repeating units represented by general formula (II):

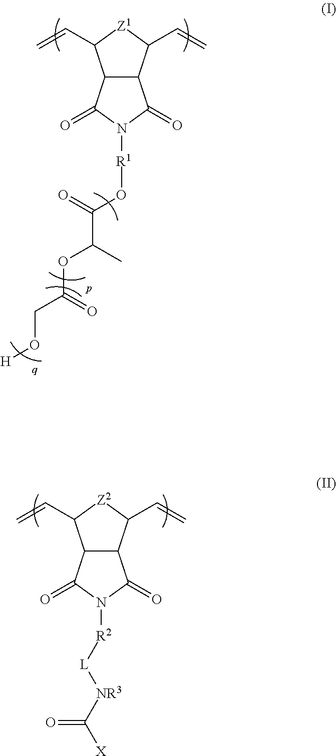

wherein
R¹ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R² is optionally substituted alkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, oligopeptides, carbohydrates, oligosaccharides, sugar, collagen, hyaluronic acid, therapeutic/drug molecules and derivatives thereof;
Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;

$$p \geq 1 ; and$$

$$q \geq 1.$$

In one embodiment, X is selected from the group consisting of RGD, SRGDS, RGDS, A5G81 (AGQWHRVSVRWGC), SVVYGLR, (IRIK)₂, (IKKI)₃, heparin oligosaccharide DP8, DP10, DP12, DP14, DP16, DGEA, (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence, hyaluronic acid and combinations thereof.

In another aspect, there is provided a method of preparing a regenerative material as disclosed herein, the method comprising:
(i) producing a bioactive PLGA filament; and
(ii) printing a bioactive PLGA material from the bioactive PLGA filament according to a design model to obtain a printed structure or part with pores patterned thereon.

In one embodiment, the step (i) of producing a bioactive PLGA filament comprises:
(i-a) providing a base PLGA powder and a bioactive PLGA copolymer;
(i-b) mixing the base PLGA powder with the bioactive PLGA copolymer to obtain a bioactive PLGA formulation; and
(i-c) extruding a bioactive PLGA filament from the formulation.

In one embodiment, the step (ii) of printing a bioactive PLGA material from the bioactive PLGA filament comprises:
(ii-a) feeding the bioactive PLGA filament into a printing apparatus;
(ii-b) applying heat to the bioactive PLGA filament to obtain a molten form of the bioactive PLGA; and
(ii-c) depositing the molten bioactive PLGA on a print bed to form a printed part or structure.

In another aspect, there is provided the regenerative material as disclosed herein for use in medicine.

In another aspect, there is provided the regenerative material as disclosed herein for use in stimulating skin and/or tissue regeneration.

In another aspect, there is provided the regenerative material as disclosed herein for use in treatment of wounds.

In another aspect, there is provided the regenerative material as disclosed herein for use in controlling and/or reducing inflammation.

In another aspect, there is provided use of a regenerative material as disclosed herein in the manufacture of a medicament for stimulating skin and/or tissue regeneration.

In another aspect, there is provided use of a regenerative material as disclosed herein in the manufacture of a medicament for treatment of wounds.

In another aspect, there is provided use of a regenerative material as disclosed herein in the manufacture of a medicament for controlling and/or reducing inflammation.

In another aspect, there is provided a method of stimulating skin and/or tissue regeneration in a subject in need thereof, the method comprising applying the regenerative material as disclosed herein to a body of the subject in need thereof.

In another aspect, there is provided a method of treating a wound, the method comprising applying the regenerative material as disclosed herein to a wound of a subject in need thereof.

In another aspect, there is provided a method of controlling and/or reducing inflammation in a subject in need thereof, the method comprising applying the regenerative material as disclosed herein to a body part of the subject in need thereof.

In one embodiment, the wound is selected from the group consisting of acute wounds, burns, incisions, excisions, superficial wounds, partial thickness wounds, full thickness wounds, chronic wounds, slow healing wounds, pressure sores, venous ulcers, diabetic ulcers, foot ulcers, bed sores and combinations thereof.

In another aspect, there is provided a medical device comprising the regenerative material as disclosed herein.

In one embodiment, the medical device is selected from the group consisting of dermal template, dermal regeneration template, skin scaffold, wound care product, wound dressing, personal care product, beauty product and combinations thereof.

### Definitions

The term “bioactive” as used herein broadly refers to the property of having a biological effect, preferably a desirable or positive biological effect on a living organism, tissue, or cell.

The term “biocompatible” as used herein broadly refers to a property of being compatible with biological systems or parts of the biological systems without substantially or significantly eliciting an adverse physiological response such as a toxic reaction, an immune reaction, an injury or the like. Such biological systems or parts include blood, cells, tissues, organs or the like.

The term “polymer” as used herein refers to a chemical compound comprising repeating units and is created through a process of polymerization. The units composing the polymer are typically derived from monomers and/or macromonomers. A polymer typically comprises repetition of a number of constitutional units.

The terms “monomer” or “macromonomer” as used herein refer to a chemical entity that may be covalently linked to one or more of such entities to form a polymer.

The term “bond” refers to a linkage between atoms in a compound or molecule. The bond may be a single bond, a double bond, or a triple bond.

In the definitions of a number of substituents below, it is stated that “the group may be a terminal group or a bridging group”. This is intended to signify that the use of the term is intended to encompass the situation where the group is a terminal group/moiety as well as the situation where the group is a linker between two other portions of the molecule. Using the term “alkyl” having 1 carbon atom as an example, it will be appreciated that when existing as a terminal group, the term “alkyl” having 1 carbon atom may mean —CH₃ and when existing as a bridging group, the term “alkyl” having 1 carbon atom may mean —CH₂— or the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “heteroalkylene” as used herein refers to alkylene having one or more —CH₂— replaced with a heteroatom selected from O, NR, Si, P or S, where R is hydrogen or alkyl as defined herein. The term “heteroalkylene” can be linear, branched or cyclic and containing up to 500 carbon atoms.

The term “alkoxy” as used herein refers to straight chain or branched alkyloxy groups. Examples include methoxy, ethoxy, n-propoxy, isopropoxy, tert-butoxy, and the like.

The term “alkoxyalkyl” as used herein is intended to broadly refer to a group containing —R—O—R′, where R and R′ are alkyl as defined herein. The group may be a terminal group or a bridging group.

The term “alkylcarbonyl” as used herein is intended to broadly refer to a group containing —R—C(═O)—, where R is alkyl as defined herein. The group may be a terminal group or a bridging group.

The term “alkylcarbonylalkyl” as used herein is intended to broadly refer to a group containing —R—C(═O)—R′, where R and R′ are alkyl as defined herein.

The group may be a terminal group or a bridging group.

The term “carboxylalkyl” as used herein is intended to broadly refer to a group containing —C(═O)—O—R, where R is alkyl as defined herein. The group may be a terminal group or a bridging group.

The term “oxycarbonylalkyl” as used herein is intended to broadly refer to a group containing —O—C(═O)—R, where R is alkyl as defined herein. The group may be a terminal group or a bridging group.

The term “alkylcarboxylalkyl” as used herein is intended to broadly refer to a group containing —R—C(═O)—O—R′, where R and R′ are alkyl as defined herein. The group may be a terminal group or a bridging group.

The term “alkoxycarbonylalkyl” as used herein is intended to broadly refer to a group containing —R—O—C(═O)—R′, where R and R′ are alkyl as defined herein. The group may be a terminal group or a bridging group.

The term “oxy” as used herein is intended to broadly refer to a group containing —O—.

The term “carbonyl” as used herein is intended to broadly refer to a group containing —C(═O)—.

The term “oxycarbonyl” as used herein is intended to broadly refer to a group containing —O—C(═O)—.

The term “carboxyl” as used herein is intended to broadly refer to a group containing —C(═O)—O—R, where R is hydrogen or an organic group.

The term “halogen” represents chlorine, fluorine, bromine or iodine. The term “halo” represents chloro, fluoro, bromo or iodo.

The term “amine group” or the like is intended to broadly refer to a group containing —NR₂, where R is independently a hydrogen or an organic group. The group may be a terminal group or a bridging group.

The term “amide group” or the like is intended to broadly refer to a group containing —C(═O)NR₂, where R is independently a hydrogen or an organic group. The group may be a terminal group or a bridging group.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The term “particle” as used herein broadly refers to a discrete entity or a discrete body. The particle described herein can include an organic, an inorganic, a composite particle or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, the term “size” when used in the context of nanoparticle can refer to the diameter of the nanoparticle although it is not limited as such. In various embodiments, when the particle is substantially spherical, the term “size” can refer to the diameter of the particle; or when the particle is substantially non-spherical, the term “size” can refer to the largest length of the particle.

The term “nano” as used herein is to be interpreted broadly to include dimensions in a nanoscale, i.e., less than about 1000 nm, about 1 nm to less than about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, or from about 1 nm to about 100 nm. Accordingly, the term “nanostructures”, “nanoparticles”, “nanomaterials” and the like as used herein may include structures that have at least one dimension in the range of no more than said range. The term “nanostructures”, “nanoparticles”, “nanomaterials” and the like as used herein may include structures that have at least one dimension that is no more than about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm.

The term “micro” as used herein is to be interpreted broadly to include dimensions from about 1 micron to about 1000 microns, about 1 micron to less than about 1000 microns, about 1 micron to about 900 microns, about 1 micron to about 800 microns, about 1 micron to about 700 microns, about 1 micron to about 600 microns, about 1 micron to about 500 microns, about 1 micron to about 400 microns, about 1 micron to about 300 microns, about 1 micron to about 200 microns, or from about 1 micron to about 100 microns.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases, symptoms and disorders. A medical condition also includes a body's response to a disease or disorder, e.g., inflammation. Those in need of such treatment include those already with a medical condition as well as those prone to getting the medical condition or those in whom a medical condition is to be prevented.

As used herein, the term “therapeutically effective amount” of a compound is intended to refer to an amount that is sufficient or capable of preventing or at least slowing down (lessening) a medical condition, such as wounds or infections/inflammations arising from wounds (e.g., acute wounds, burns, incisions, excisions, superficial wounds, partial thickness wounds, full thickness wounds, chronic wounds, slow healing wounds, pressure sores, venous ulcers, diabetic ulcers, foot ulcers, bed sores or the like). Dosages and administration of compounds, compositions and formulations of the present disclosure may be determined by one of ordinary skill in the art of clinical pharmacology or pharmacokinetics. An effective amount of the active agent of the present disclosure to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect.

The term “subject” is intended to broadly refer to any animal, such as a mammal, and including humans. Exemplary subjects include but are not limited to humans and non-human primates. The term “subject” as used herein also includes patients and non-patients. The term “patient” refers to individuals suffering or are likely to suffer from a medical condition such as wounds (e.g., acute wounds, burns, incisions, excisions, superficial wounds, partial thickness wounds, full thickness wounds, chronic wounds, slow healing wounds, pressure sores, venous ulcers, diabetic ulcers, foot ulcers, bed sores or the like), while “non-patients” refer to individuals not suffering and are likely to not suffer from the medical condition. “Non-patients” include healthy individuals, non-diseased individuals and/or an individual free from the medical condition. As used herein, the term “mammal” includes vertebrate such as a human or a large veterinary mammal (e.g., horses, cattle, deer, sheep, llamas, goats, pigs).

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

It will also be appreciated that where priority is claimed to an earlier application, the full contents of the earlier application is also taken to form part of the present disclosure and may serve as support for embodiments disclosed herein.

### DESCRIPTION OF EMBODIMENTS

Exemplary, non-limiting embodiments of a regenerative material, a bioactive poly(lactic-co-glycolic acid) (PLGA) material, related printed structure, a method of preparing said regenerative material and/or bioactive PLGA material and related methods/uses thereto are disclosed hereinafter.

Bioactive Poly(lactic-co-glycolic acid) (PLGA) Material

There is provided a bioactive poly(lactic-co-glycolic acid) (PLGA) material. Advantageously, in various embodiments, the material is suitable for promoting or stimulating connective tissue regeneration such as skin regeneration and/or tissue regeneration. Accordingly, in various embodiments, the material is a regenerative material. Advantageously, in various embodiments, the material is also suitable for promoting or stimulating wound healing/regeneration/regrowth/repair/closure/treatment as well as in methods of treating wounds. The material may also be a material that is suitable for use in controlling and/or reducing inflammation. In various embodiments, by controlling and/or reducing inflammation, embodiments of the material may also be suitable for use in reducing risk of scarring (which may result due to prolonged inflammation). Advantageously, the material may serve as a dermal template, dermal regeneration template, dermal substitute, skin substitute, skin scaffold, tissue scaffold, wound care, wound dressing, personal care and/or beauty product to treat, repair and/or heal wounds. In various embodiments, the material overcomes or at least ameliorates one or more of the inherent issues of conventional wound care products as described above.

In various embodiments, the material comprises a bioactive poly(lactic-co-glycolic acid) (PLGA) copolymer. In various embodiments, the material further comprises a base polymer. In various embodiments, the base polymer is a synthetic polymer. In various embodiments, the base polymer is a medical grade polymer. In various embodiments, the base polymer is biodegradable, allowing the material to be broken down naturally. In various embodiments, the base polymer comprises PLGA. The bioactive PLGA copolymer may be blended/mixed with the base PLGA.

In various embodiments, the bioactive PLGA material comprises/consists essentially of/consists of:
(i) a base poly(lactic-co-glycolic acid) (PLGA);
(ii) a bioactive poly(lactic-co-glycolic acid) (PLGA) copolymer; and
(iii) pores/channels/micropores/microchannels patterned on said material.

In various embodiments, the term “poly(lactic-co-glycolic acid)” comprises and/or may be used interchangeably with the terms “poly(lactide-co-glycolide)”, “poly(D-lactide-co-glycolide)”, “poly(L-lactide-co-glycolide)”, “poly(D,L-lactide-co-glycolide)” or the like. In various embodiments, PLGA comprises medical grade PLGA.

In various embodiments, the material comprises pores patterned on said material. In various embodiments, the material comprises a plurality of (or one or more) pores patterned on said material. Advantageously, due to the presence of pores, embodiments of the material possess good/high permeability to fluid (e.g., air and moisture), allowing fluid penetration/exchange/transfer through the material. In various embodiments, presence of the pores patterned on the material allows moisture to be extracted/removed/transferred away from a wound. In various embodiments, presence of the pores patterned on the material allows oxygen to be supplied to the wound. Even more advantageously, the pore design helps in providing a conducive and/or healthy environment to wound healing by preventing excess fluid from trapping/accumulating within the wound, while at the same time, allowing oxygen to penetrate and reach the wound. In various embodiments, the pore design of the bioactive PLGA material allows for both moisture extraction and oxygen penetration, thereby making the material suitable for use in wound care/healing applications. In various embodiments, the material is suitable for use in improving/enhancing healing outcomes, and in reducing the risk of scarring (e.g., permanent scarring).

In various embodiments, each pore comprises a polygonal shape, polygonal-shaped pore structure or polygonal morphology. In various embodiments, each pore comprises a triangle-shaped, square-shaped, quadrilateral-shaped or hexagonal-shaped pore structure or polygonal morphology. In various embodiments, each pore comprises a polygonal shape having n sides where n 3 such as a triangular shape (i.e. n=3), square shape (i.e. n=4), quadrilateral shape (i.e. n=4), pentagonal shape (i.e. n=5), hexagonal shape (i.e. n=6), heptagonal shape (i.e. n=7), octagonal shape (i.e. n=8), nonagonal shape (i.e. n=9) or decagonal shape (i.e. n=10). In various embodiments, n 3, 4, 5, 6, 7, 8, 9, or 10.

In various embodiments, each pore has a minimum circumscribed circle diameter of about 100.0 μm. In various embodiments, each pore has a circumscribed circle diameter of at least about 100.0 μm, at least about 105.0 μm, at least about 110.0 μm, at least about 115.0 μm, at least about 120.0 μm, at least about 125.0 μm, at least about 130.0 μm, at least about 135.0 μm, at least about 140.0 μm, at least about 145.0 μm, at least about 150.0 μm, at least about 155.0 μm, at least about 160.0 μm, at least about 165.0 μm, at least about 170.0 μm, at least about 175.0 μm, at least about 180.0 μm, at least about 185.0 μm, at least about 190.0 μm, at least about 195.0 μm, at least about 200.0 μm, at least about 205.0 μm, at least about 210.0 μm, at least about 215.0 μm, at least about 220.0 μm, at least about 225.0 μm, at least about 230.0 μm, at least about 235.0 μm, at least about 240.0 μm, at least about 245.0 μm, at least about 250.0 μm, at least about 255.0 μm, at least about 260.0 μm, at least about 265.0 μm, at least about 270.0 μm, at least about 275.0 μm, at least about 280.0 μm, at least about 285.0 μm, at least about 290.0 μm, at least about 295.0 μm, or at least about 300.0 μm. It will be appreciated that as the pore size is governed by design requirements (e.g., the pore size may be customised/adjusted as desired to suit a particular application/requirement), there is no limit to the pore size (e.g., circumscribed circle diameter of the pore). In some embodiments, the pores are designed to be as small as possible in order to maximize the amount of substances (e.g., bioactive moiety) that may be contained within the material for interaction with a wound.

In various embodiments, the distance between the pores is from about 100.0 μm to about 300.0 μm. For example, the distance between a pore from another pore may be from about 100.0 μm to about 300.0 μm, from about 105.0 μm to about 295.0 μm, from about 110.0 μm to about 290.0 μm, from about 115.0 μm to about 285.0 μm, from about 120.0 μm to about 280.0 μm, from about 125.0 μm to about 275.0 μm, from about 130.0 μm to about 270.0 μm, from about 135.0 μm to about 265.0 μm, from about 140.0 μm to about 260.0 μm, from about 145.0 μm to about 255.0 μm, from about 150.0 μm to about 250.0 μm, from about 155.0 μm to about 245.0 μm, from about 160.0 μm to about 240.0 μm, from about 165.0 μm to about 235.0 μm, from about 170.0 μm to about 230.0 μm, from about 175.0 μm to about 225.0 μm, from about 180.0 μm to about 220.0 μm, from about 185.0 μm to about 215.0 μm, from about 190.0 μm to about 210.0 μm, from about 195.0 μm to about 205.0 μm, or about 200.0 μm.

In various embodiments, the material comprises a buffer zone/area surrounding the pores/channels/micropores/microchannels. In various embodiments, the buffer zone/area is substantially devoid of pores/channels/micropores/microchannels and/or has substantially lesser pores/channels/micropores/microchannels than the area of the material it surrounds.

In various embodiments, the buffer zone/area forms the border or perimeter of the material.

In various embodiments, the buffer zone/area has a width of from about 1 mm to about 10 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, or about 10.0 mm.

In various embodiments, the material has a thickness/height/depth of from about 0.05 mm to about 0.60 mm, from about 0.06 mm to about 0.59 mm, from about 0.07 mm to about 0.58 mm, from about 0.08 mm to about 0.57 mm, from about 0.09 mm to about 0.56 mm, from about 0.10 mm to about 0.55 mm, from about 0.11 mm to about 0.54 mm, from about 0.12 mm to about 0.53 mm, from about 0.13 mm to about 0.52 mm, from about 0.14 mm to about 0.51 mm, from about 0.15 mm to about 0.50 mm, from about 0.16 mm to about 0.49 mm, from about 0.17 mm to about 0.48 mm, from about 0.18 mm to about 0.47 mm, from about 0.19 mm to about 0.46 mm, from about 0.20 mm to about 0.45 mm, from about 0.21 mm to about 0.44 mm, from about 0.22 mm to about 0.43 mm, from about 0.23 mm to about 0.42 mm, from about 0.24 mm to about 0.41 mm, from about 0.25 mm to about 0.40 mm, from about 0.26 mm to about 0.39 mm, from about 0.27 mm to about 0.38 mm, from about 0.28 mm to about 0.37 mm, from about 0.29 mm to about 0.36 mm, from about 0.30 mm to about 0.35 mm, from about 0.31 mm to about 0.34 mm, or from about 0.32 mm to about 0.33 mm.

In various embodiments, the material has an infill density of from about 70.0% to about 100.0%, from about 71.0% to about 99.9%, from about 72.0% to about 99.8%, from about 73.0% to about 99.7%, from about 74.0% to about 99.6%, from about 75.0% to about 99.5%, from about 76.0% to about 99.0%, from about 77.0% to about 98.0%, from about 78.0% to about 97.0%, from about 79.0% to about 96.0%, from about 80.0% to about 95.0%, from about 81.0% to about 94.0%, from about 82.0% to about 93.0%, from about 83.0% to about 92.0%, from about 84.0% to about 91.0%, from about 85.0% to about 90.0%, from about 86.0% to about 89.0%, or from about 87.0% to about 88.0%.

In various embodiments, the ratio of the base PLGA to bioactive PLGA copolymer present in the material is from about 60.0-99.9:0.1-40.0.

In various embodiments, the material comprises from about 60.0 wt % to about 99.9 wt %, from about 61.0 wt % to about 99.8 wt %, from about 62.0 wt % to about 99.7 wt %, from about 63.0 wt % to about 99.6 wt %, from about 64.0 wt % to about 99.5 wt %, from about 65.0 wt % to about 99.0 wt %, from about 66.0 wt % to about 98.5 wt %, from about 67.0 wt % to about 98.0 wt %, from about 68.0 wt % to about 97.0 wt %, from about 69.0 wt % to about 96.0 wt %, from about 70.0 wt % to about 95.0 wt %, from about 71.0 wt % to about 94.0 wt %, from about 72.0 wt % to about 93.0 wt %, from about 73.0 wt % to about 92.0 wt %, from about 74.0 wt % to about 91.0 wt %, from about 75.0 wt % to about 90.0 wt %, from about 76.0 wt % to about 89.0 wt %, from about 77.0 wt % to about 88.0 wt %, from about 78.0 wt % to about 87.0 wt %, from about 79.0 wt % to about 86.0 wt %, from about 80.0 wt % to about 85.0 wt %, from about 81.0 wt % to about 84.0 wt %, or from about 82.0 wt % to about 83.0 wt % of the base PLGA.

In various embodiments, the material comprises from about 0.1 wt % to about 40.0 wt %, from about 0.2 wt % to about 39.0 wt %, from about 0.3 wt % to about 38.0 wt %, from about 0.4 wt % to about 37.0 wt %, from about 0.5 wt % to about 36.0 wt %, from about 1.0 wt % to about 35.0 wt %, from about 1.5 wt % to about 34.0 wt %, from about 2.0 wt % to about 33.0 wt %, from about 3.0 wt % to about 32.0 wt %, from about 4.0 wt % to about 31.0 wt %, from about 5.0 wt % to about 30.0 wt %, from about 6.0 wt % to about 29.0 wt %, from about 7.0 wt % to about 28.0 wt %, from about 8.0 wt % to about 27.0 wt %, from about 9.0 wt % to about 26.0 wt %, from about 10.0 wt % to about 25.0 wt %, from about 11.0 wt % to about 24.0 wt %, from about 12.0 wt % to about 23.0 wt %, from about 13.0 wt % to about 22.0 wt %, from about 14.0 wt % to about 21.0 wt %, from about 15.0 wt % to about 20.0 wt %, from about 16.0 wt % to about 19.0 wt %, or from about 17.0 wt % to about 18.0 wt % of the bioactive PLGA copolymer.

In various embodiments, the material comprises a printed structure or part. For example, the printed structure or part may be a three-dimensional (3D) printed structure or part. In various embodiments therefore, there is provided a poly(lactic-co-glycolic acid) (PLGA) three-dimensional printed structure.

In various embodiments, the material is printed (e.g., three-dimensional printed) via fused filament fabrication (FFF), fused deposition modelling (FDM) or the like. In various embodiments, the material is obtained via FFF-based or FDM-based 3D printing. Advantageously, in various embodiments, filament extrusion and 3DP by Fused Filament Fabrication methods do not denature the biomolecules present in the material.

In various embodiments, the material is suitable for use as a dermal template, dermal regeneration template, dermal substitute, skin substitute, skin scaffold, tissue scaffold, wound care, personal care and/or beauty product.

In various embodiments, there is provided a medical device comprising the bioactive PLGA material/printed structure or part as disclosed herein. In various embodiments, there is provided a wound dressing comprising the bioactive PLGA material/printed structure or part as disclosed herein.

In various embodiments, the material/printed structure has one or more of the following properties: oxygen permeable; moisture/fluid/water permeable; flexible (i.e. capable of conforming to surface/site that it is applied to/on); thermally stable (i.e. capable of undergoing high temperatures e.g., melt processing); mechanically stable (i.e. does not warp and/or thin substantially); biodegradable; bioresorbable; and/or biocompatible.

In various embodiments, the material is biodegradable and/or capable of being broken down naturally. Advantageously, the biodegradable property of the material eliminates the need/requirement for removal of the material (e.g., scaffold or template removal) after use.

Bioactive Poly(lactic-co-glycolic acid) (PLGA) Copolymer

In various embodiments, the bioactive PLGA material comprises/consists essentially of/consists of a bioactive PLGA copolymer with a poly(norbornene-dicarboximide) backbone having one or more repeating units represented by general formula (I) and one or more repeating units represented by general formula (II):

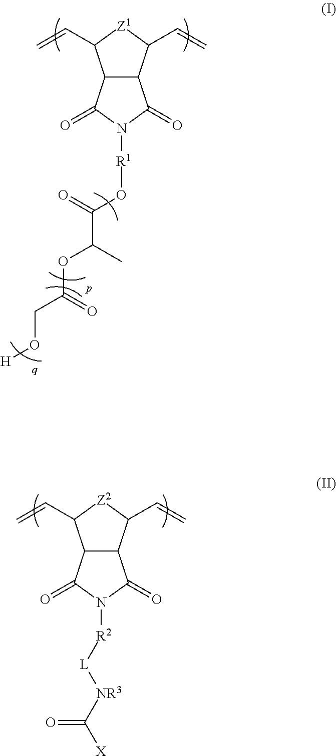

wherein
R¹ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R² is optionally substituted alkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, oligopeptides, carbohydrates, oligosaccharides, sugar, collagen, hyaluronic acid, therapeutic/drug molecules and derivatives thereof;
Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;

$$p \geq 1 ; and$$

$$q \geq 1.$$

In various embodiments, the repeating unit(s) represented by general formula (I) possess good mechanical strength/hardness. In various embodiments, the repeating unit(s) represented by general formula (II) and/or moiety X possess bioactivity, biocompatibility and/or biodegradability. In various embodiments, the repeating unit represented by general formula (I) has a higher mechanical strength than the repeating unit represented by general formula (II) and/or moiety X. Advantageously, the presence of repeating units represented by general formulae (I) and (II) in the bioactive PLGA copolymer imparts both bioactivity and mechanical strength to the copolymer, leading to a mechanically strong bioactive copolymer. In various embodiments, the copolymer may also be biocompatible and/or biodegradable. Accordingly, in various embodiments, the copolymer is capable of being classified as a biomaterial. Advantageously, due to the presence of synthetic and bioactive side chains, the bioactive PLGA copolymer may also have a high thermal stability than conventional biomolecules such as peptides, proteins, carbohydrates or glycosaminoglycans. Even more advantageously, the thermal stability of the bioactive hyaluronic acid-based synthetic copolymer allows for embodiments of the copolymer to be suitable for processing at high temperatures or even harsh material processing such as melt processing/extrusion >100° C., making the copolymer ideal/attractive for use in applications such as biomedical devices. In various embodiments, the repeating unit(s) represented by general formula (I) is substantially or completely non-bioactive, or at least less bioactive than the repeating unit(s) represented by general formula (II) and/or bioactive moiety X.

In various embodiments, L is a polymeric linker that links the bioactive moiety X to the poly(norbornene) backbone. Advantageously, L is designed to be adjustable and/or customizable based on the size of the bioactive moiety X and the size of the synthetic polymer (i.e. PLGA). The molecular weight and/or length of the polymeric linker L may be customized to suit the molecular weight and/or length of the bioactive moiety X and the synthetic polymer (i.e. PLGA), depending on the application the copolymer is to be used for. In various embodiments, physical properties of the copolymer can be changed/tuned/customized depending on the length of L (e.g., PEG chain). For example, in skin scaffolds, shorter polymeric (i.e. PLGA) side chains may be preferred for fast degradation. In various embodiments, for applications in dressings, or particularly non-biodegradable non-woven fibers which require thermal stability and/or mechanical strength properties, low molecular weight may be preferred for PLGA due to its poor solubility in common solvents.

In various embodiments, the molecular weight and/or length of the polymeric linker L is selected such that the overall molecular size of the repeating unit represented by general formula (I) is similar/comparable to the molecular size of the repeating unit represented by general formula (II). For example, if PLGA having a molecular weight of 4,000 is selected and a peptide having a molecular weight of from about 400 to about 500 is selected as the choice of bioactive moiety X, then L may be designed to comprise a molecular weight of about 3,400. It will be appreciated that in various embodiments, it is the length of L that gets adjusted to match the molecular weight of general formula (I) to molecular weight of general formula (II).

In various embodiments, the molecular weight of general formula (I) is comparable/substantially similar with/to the molecular weight of general formula (II). In various embodiments, the molecular weight of general formula (I) does not differ from the molecular weight of general formula (II) by more than 30% of the molecular weight of general formula (II) or vice versa. For example, the molecular weight of general formula (I) may be at most about 30% more or at most 30% less than the molecular weight of general formula (II) or vice versa. The molecular weight of general formula (I) may not differ from the molecular weight of general formula (II) by more than about 30%, more than about 25%, more than about 20%, more than about 15%, more about 10%, more than about 5%, more than about 4%, more than about 3%, more than about 2%, or more than about 1% of the molecular weight of general formula (II) or vice versa. In various embodiments, the molecular weight of general formula (I) does not differ from the molecular weight of general formula (II) by more than about 20% of the molecular weight of general formula (II) or vice versa. For example, the molecular weight of general formula (I) may be at most about 20% more or at most 20% less than the molecular weight of general formula (II) or vice versa. Advantageously, as the bioactive moiety bearing repeating unit has a molecular size/weight/length that is similar to that of the synthetic polymer (i.e. PLGA) bearing repeating unit, the length of the bioactive moiety X is extended, thereby allowing X to be “visible”, available for binding to cells or accessible to its targeted physiological site for desired bioactivity, i.e. not buried in a sea/matrix of synthetic polymers (i.e. PLGA).

In various embodiments, the molecular weight of general formula (I) is from about 100 to about 15,000, from about 200 to about 14,000, from about 300 to about 13,000, from about 400 to about 12,000, from about 500 to about 11,000, from about 1,000 to about 10,000, from about 1,500 to about 9,500, from about 2,000 to about 9,000, from about 2,500 to about 8,500, from about 3,000 to about 8,000, from about 3,500 to about 7,500, from about 4,000 to about 7,000, from about 4,500 to about 6,500, from about 5,000 to about 6,000 or about 5,500.

In various embodiments, the molecular weight of general formula (II) is about 15,000, about 14,000, about 13,000 or at least about 12,000. In various embodiments, the molecular weight of general formula (II) is from about 100 to about 15,000, from about 200 to about 14,000, from about 300 to about 13,000, from about 400 to about 12,000, from about 500 to about 11,000, from about 1,000 to about 10,000, from about 1,500 to about 9,500, from about 2,000 to about 9,000, from about 2,500 to about 8,500, from about 3,000 to about 8,000, from about 3,500 to about 7,500, from about 4,000 to about 7,000, from about 4,500 to about 6,500, from about 5,000 to about 6,000 or about 5,500.

In various embodiments, the total molecular weight of general formula (I) and general formula (II) is kept to about 300,000, no more than about 300,000, no more than about 200,000, no more than about 100,000, no more than about 90,000, no more than about 80,000, no more than about 70,000, no more than about 60,000, no more than about 50,000, no more than about 45,000, no more than about 40,000, no more than about 35,000, no more than about 30,000, no more than about 25,000, no more than about 20,000, or no more than about 15,000 to facilitate copolymerisation.

In various embodiments, the ratio of the number of repeating units represented by general formula (I) to the number of repeating units represented by general formula (II) in the bioactive PLGA copolymer is from about 1:1 to about 1:100, from about 1:2 to about 1:99, from about 1:3 to about 1:98, from about 1:4 to about 1:97, from about 1:5 to about 1:96, from about 1:6 to about 1:95, from about 1:7 to about 1:90, from about 1:8 to about 1:85, from about 1:9 to about 1:80, from about 1:10 to about 1:75, from about 1:15 to about 1:70, from about 1:20 to about 1:65, from about 1:25 to about 1:60, from about 1:30 to about 1:55, from about 1:35 to about 1:50, or from about 1:40 to about 1:45.

In various embodiments, the number of repeating units represented by general formula (I) in the bioactive PLGA copolymer is from about 10 to about 1,000. For example, the number of repeating units represented by general formula (I) in the bioactive PLGA copolymer may be from about 10 to about 1,000, from about 50 to about 950, from about 100 to about 900, from about 150 to about 850, from about 200 to about 800, from about 250 to about 750, from about 300 to about 700, from about 350 to about 650, from about 400 to about 600, from about 450 to about 550, or about 500.

In various embodiments, the number of repeating units represented by general formula (II) in the bioactive PLGA copolymer is from about 10 to about 1,000. For example, the number of repeating units represented by general formula (II) in the bioactive PLGA copolymer may be from about 10 to about 1,000, from about 50 to about 950, from about 100 to about 900, from about 150 to about 850, from about 200 to about 800, from about 250 to about 750, from about 300 to about 700, from about 350 to about 650, from about 400 to about 600, from about 450 to about 550, or about 500.

In various embodiments, L is hydrophilic. As L is adjustable, the hydrophilicity of the repeating unit represented by general formula (II) and also the overall hydrophilicity of the bioactive PLGA copolymer may be adjusted as desired. Advantageously, the presence of L increases the hydrophilicity of the repeating unit represented by general formula (II) and also the overall hydrophilicity of the bioactive synthetic copolymer. Even more advantageously, the presence of L increases the hydrophilicity of the bioactive synthetic copolymer, therefore softening the synthetic polymeric chains which are hydrophobic, making the copolymer less stiff after processing. It will be appreciated by a person skilled in the art that, bioactive moieties and synthetic polymers are typically mutually incompatible as the individual bioactive moiety is generally hydrophilic while synthetic polymer is generally hydrophobic. Advantageously, L in repeating unit represented by general formula (II) is also used to extend the chain length of the bioactive moiety X attached at the end of L.

In various embodiments, L is amorphous. Advantageously, the presence of L increases the amorphousness and/or decreases the crystallinity of the bioactive synthetic copolymer, making the copolymer useful for crafting softer, flexible or less stiff plastics.

In various embodiments, L is a heteroalkylene having at least 20 carbon atoms, at least 30 carbon atoms, at least 40 carbon atoms, at least 50 carbon atoms, at least 60 carbon atoms, at least 70 carbon atoms, at least 80 carbon atoms, at least 90 carbon atoms, at least 100 carbon atoms, at least 150 carbon atoms, at least 200 carbon atoms, at least 250 carbon atoms or at least 300 carbon atoms. In various embodiments, L is C₂₀-C₃₀₀ heteroalkylene or a heteroalkylene having from 20 carbon atoms to 300 carbon atoms.

In various embodiments, L has a number average molecular weight of between about 500 and about 7,000. L may have a number average molecular weight of about 600, about 700, about 800, about 900, about 1,000, about 1,500, about 2,000, about 2,500, about 3,000, about 3,500, about 4,000, about 4,500, about 5,000, about 5,500, about 6,000, about 6,500 or about 7,000. In various embodiments, when X comprises a small bioactive moiety, the molecular weight of L may be adjusted to about 7,000 so that the total molecular weight of general formula (I) and general formula (II) is kept to no more than about 10,000. In various embodiments, the number average molecular weight of L is from about 1,000 to about 6,000.

In various embodiments, the heteroatom in L is O. In various embodiments, L is polyalkylene glycol, e.g., poly(C₂-C₄ alkylene glycol). L may be selected from the group consisting of polyethylene glycol (PEG), polypropylene glycol (PPG), polytetramethylene glycol (PTMG), polybutylene glycol (PBG) and the like, and combinations thereof. Advantageously, the use of a polyalkylene glycol such as PEG can increase hydrophilicity of the copolymer. In various embodiments, polyalkylene glycol such as PEG are used as spacers, linkers or linking groups in the overall polymers, instead of as terminal groups.

In various embodiments, L is polyalkylene glycol having at least about 10 repeating units, at least about 15 repeating units, at least about 20 repeating units, at least about 21 repeating units, at least about 22 repeating units, at least about 23 repeating units, at least about 24 repeating units, at least about 25 repeating units, at least about 30 repeating units, at least about 40 repeating units, at least about 50 repeating units, at least about 60 repeating units, at least about 70 repeating units, at least about 80 repeating units, at least about 90 repeating units, at least about 100 repeating units, at least about 150 repeating units, at least about 200 repeating units, or at least about 250 repeating units. In various embodiments, L comprises from about 10 monomers/repeating units to about 250 monomers/repeating units. Unlike conventional polymers which uses a short PEG chain, embodiments of the bioactive synthetic copolymer disclosed herein incorporate a long polyalkylene glycol chain of at least 21 repeating units at L.

In various embodiments, L comprises from about 10 monomers/repeating units to about 250 monomers/repeating units. For example, L is selected from the group consisting of PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₂₀₀₀, PEG₂₅₀₀, PEG₃₀₀₀, PEG₃₅₀₀, PEG₄₀₀₀, PEG₄₅₀₀, PEG₅₀₀₀, PEG₅₅₀₀, PEG₆₀₀₀, PEG₆₆₀₀ and mixtures thereof.

In various embodiments, X is coupled to the poly(norbornene dicarboximide) backbone through a carboxylic acid functionality in the following arrangement: —R¹-L-NR³—C(═O)—X. Advantageously, by linking X through a carboxylic acid functionality, amine terminal group(s) in X is/are free up for delivering its bioactivity, therefore ensuring the bioavailability of X. It will be appreciated that as amine group(s) confer bioactivity, exhausting up amine groups in bioactive moieties for polymer binding may be undesirable.

In various embodiments, X is coupled to the poly(norbornene dicarboximide) backbone via peptide/amide linkage, i.e. —NR³—C(═O)—. Advantageously, the bioactive PLGA copolymer disclosed herein is considerably stronger and/or stable than conventional polymers that contain ester linkages. Without being bound by theory, it is believed that amide linkages are stronger than ester linkages because ester linkages are more prone to hydrolysis, which may release bioactive moieties into the bloodstream, leading to a premature metabolism of bioactive moieties. Advantageously, the presence of an amide linkage prevents the bioactive moieties from breaking off from the polymer chain, therefore ensuring the bioavailability of the bioactive moieties. It will be appreciated that the active site for bioactivity (e.g., cell binding) is at bioactive moieties in general formula (II).

In various embodiments, one or more of H atoms in alkyl, alkenyl, alkynyl, alkoxyalkyl, alkylcarbonyl and alkylcarbonylalkyl is/are optionally replaced by hydroxy, hydroxyalkyl, halogen, haloalkyl, cyano, cyanoalkyl and nitro.

In various embodiments, R¹ is selected from C₁-C₂₀ alkyl, C₂-C₂₀ alkenyl, C₂-C₂₀ alkynyl, C₁-C₂₀ alkoxyalkyl, C₂-C₂₀ alkylcarbonyl or C₃-C₂₀ alkylcarbonylalkyl. The C₁-C₂₀ alkyl substituents may be straight or branched substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, the like or combinations thereof.

In various embodiments, R² is selected from straight or branched C₁-C₂₀ alkyl. The C₁-C₂₀ alkyl substituents may be straight or branched substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, the like or combinations thereof. R² may be straight or branched C₁-C₄alkyl substituents. In various embodiments, the length of R² is the same as the length of a repeating unit in L. For example, if L is poly(butylene glycol), then R² is butyl. In another example, if L is poly(ethylene glycol), then R² is ethyl. It will be appreciated that in various embodiments, R² is carefully designed to match L.

In various embodiments, R³ is selected from H, C₁-C₂₀ alkyl, C₂-C₂₀ alkenyl or C₂-C₂₀ alkynyl.

In various embodiments, Z¹ and Z² are each independently selected from CH₂, O, NH, SiR^{a}R^{b}, PR^{a} or S. The poly(norbornene) backbone may be selected from the group consisting of poly(norbornene-imide), poly(norbornene-dicarboximide), poly(5-norbornene-2,3-dicarboximide), poly(7-oxanorbornene), poly(oxanorbornene-imide), poly(oxanorbornene-dicarboximide) and the like. In various embodiments, Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, C₁-C₂₀ alkyl, C₁-C₂₀ alkenyl and C₁-C₂₀ alkynyl. In various embodiments, Z¹ is CH₂. In various embodiments, Z² is CH₂.

In various embodiments, X comprises a bioactive moiety selected from proteins, peptides, carbohydrates, therapeutic/drug molecules and derivatives thereof. In various embodiments, proteins, peptides, carbohydrates or therapeutic/drug molecules derivatives thereof include proteins, peptides, carbohydrates or therapeutic/drug molecules that are or have been optionally modified to contain one carboxylic acid terminal group. In some embodiments, the bioactive moiety contains only one carboxylic acid terminal group.

In various embodiments, the bioactive moiety comprises a monocarboxylic acid. Advantageously, in some embodiments, the use of a bioactive moiety having a monocarboxylic acid terminal group may reduce/avoid the possibility of an undesirable crosslinking as compared to the case of using more than one carboxylic acid. In some embodiments therefore, the bioactive moiety X is substantially devoid of more than one carboxylic acid terminal group, for e.g., a dicarboxylic acid or tricarboxylic acid.

In various embodiments, X comprises protein or peptide. X may be a peptide sequence, laminin-derived peptide, integrin binding peptide, cell-penetrating peptide, collagen mimics or collagen fragments. In various embodiments, X comprises from 2 to 50 amino acid residues, from 2 to 40 amino acid residues or from 2 to 20 amino acid residues in any sequence. In various embodiments, X comprises 50 amino acid residues, 40 amino acid residues, 30 amino acid residues, 25 amino acid residues, 20 amino acid residues, 15 amino acid residues, 10 amino acid residues, 9 amino acid residues, 8 amino acid residues, 7 amino acid residues, 6 amino acid residues, 5 amino acid residues, 4 amino acid residues or 3 amino acid residues in any sequence. The amino acid residues may be selected from the group consisting of glycine, alanine, valine, leucine, isoleucine, methionine, proline, phenylalanine, tryptophan, asparagine, glutamine, glycine, serine, threonine, serine, asparagine, glutamine, tyrosine, cysteine, lysine, arginine, histidine, aspartic acid and glutamic acid. In various embodiments, X is a peptide sequence comprising 3 to 20 natural amino acids. X may be integrin binding peptide selected from the group consisting of arginine-glycine-aspartic acid (RGD), SRGDS and RGDS; laminin-derived peptide A5G81 (AGOWHRVSVRWGC); osteopontin derived peptides SVVYGLR; and cell-penetrating/antimicrobial peptide selected from (IRIK)₂ or (IKKI)₃. In various embodiments, X is a collagen sequence comprising 3 to 20 units of glycine (G), proline (P) and hydroxyproline (Hyp) in any sequence or permutation. X may be collagen fragment having a (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence or collagen mimic DGEA.

In various embodiments, X comprises non-type I collagen. In various embodiments, the material is a non-animal derived material. Advantageously, by using non-type I collagen, the material reduces and/or eliminates risk of bacteria infection (which may otherwise result from the use of Type I collagen).

In various embodiments, X comprises carbohydrate or sugar. In various embodiments, X comprises monosaccharide, disaccharide, oligosaccharide or polysaccharide. In various embodiments, X comprises from 2 to 50 saccharide units, from 2 to 40 saccharide units, from 2 to 20 saccharide units or from 10 to 14 saccharide units. In various embodiments, X comprises 50 saccharide units, 40 saccharide units, 30 saccharide units, 25 saccharide units, 20 saccharide units, 15 saccharide units, 14 saccharide units, 13 saccharide units, 12 saccharide units, 11 saccharide units, 10 saccharide units, 9 saccharide units, 8 saccharide units, 7 saccharide units, 6 saccharide units, 5 saccharide units, 4 saccharide units or 3 saccharide units or 2 saccharide units. X may be heparin sulfate (HS) or glycosaminoglycans (GAGs). In various embodiments, X is heparin sulfate/oligosaccharide selected from the group consisting of DP8, DP10, DP12, DP14 and DP16. In various embodiments, X is hyaluronic acid which is the simplest form of glycosaminoglycan (GAG). For example, X may be hyaluronic acid comprising from 8 to 20 saccharide units, from 8 to 13 saccharide units, about 8 saccharide units, about 9 saccharide units, about 10 saccharide units, about 11 saccharide units, about 12 saccharide units, about 13 saccharide units, about 14 saccharide units, about 15 saccharide units, about 16 saccharide units, about 17 saccharide units, about 18 saccharide units, about 19 saccharide units, or about 20 saccharide units.

In various embodiments, the term “hyaluronic acid” comprises and/or may be used interchangeably with the term “hyaluronic acid and/or derivatives thereof”, “hyaluronic acid”, “hyaluronan”, “derivatives of hyaluronic acid”, “conjugate base of hyaluronic acid” and ““hyaluronate”.

In various embodiments, X is chemically coupled to the rest of general formula (II) via its hydroxy group. For example, when X is carbohydrate/saccharide, oxidation and/or reductive amination reactions may be performed on the carbohydrate's hydroxy for linking X to general formula (II). —CH₂OH on the saccharide may be oxidised to —C(═O)H, which subsequently undergoes reductive amination using the —NH₂ terminal on L to create a peptide linkage.

In various embodiments, X comprises a carbohydrate/saccharide that contained or has been modified to contain one carboxylic acid terminal group. Modification by one or more chemical reaction(s) such as oxidation may be performed on the carbohydrate/saccharide to create a carboxylic acid group. In various embodiments, modification is performed on a hydroxyl group that is originally present in the carbohydrate/saccharide. In various embodiments, —CH₂OH on the carbohydrate/saccharide is oxidized completely to —C(═O)OH, which subsequently reacts with a —NH₂ terminal on L to create a peptide linkage that links the carbohydrate/saccharide to the rest of general formula (II): X—C(═O)—NH-L-. It will be appreciated, however, that no modification to the carbohydrate/saccharide may be required/necessary if a carboxylic acid is naturally present in the carbohydrate/saccharide.

In various embodiments, X comprises therapeutic/drug molecule. In various embodiments, X comprises antibiotic, antimicrobial, antibacterial, blood thinning agents or anti-inflammatory agents. X may be penicillin, amoxicillin, amphotericin, ciprofloxacin (CIF), atorvastatin, aspirin or aminoglycoside-based molecules selected from streptomycin, ribostamycin or gentamycin. It will be appreciated that X may be any therapeutic or drug molecule that contains a carboxylic acid group.

In various embodiments, X is chemically coupled to the rest of general formula (II) via one of its chemical moiety selected from the group consisting of —COOH, —CH₂OH, —CH₂NH₂ and ═CHNH₂. For example, —CH₂NH₂ or ═CHNH₂ on the drug molecule may be coupled to a small dicarboxylic acid before reacting with a —NH₂ terminal on L to create a peptide linkage that links the drug molecule to the rest of general formula (II): X—C(═O)—NH-L-.

In various embodiments, X comprises a therapeutic/drug molecule that contained or has been modified to contain one carboxylic acid terminal group. Modification by one or more chemical reaction(s) such as oxidation may be performed on the therapeutic/drug molecule to create a carboxylic acid group. In various embodiments, modification is performed on a hydroxyl group that is originally present in the therapeutic/drug molecule. For example, in various embodiments when X is ribostamycin or gentamycin, —CH₂OH on the drug molecule is oxidized completely to —C(═O)OH, which subsequently reacts with a —NH₂ terminal on L to create a peptide linkage that links the drug molecule to the rest of general formula (II): X—C(═O)—NH-L-. It will be appreciated, however, that no modification to the therapeutic/drug molecule may be required/necessary if a carboxylic acid is already present in the therapeutic/drug molecule.

In various embodiments, the bioactive moiety is or has been modified to contain one carboxylic acid terminal group. For example, if a carboxylic acid terminal group is absent in a carbohydrate or therapeutic/drug molecule, the carbohydrate or therapeutic/drug molecule may be modified to add a carboxylic acid at one of the carbohydrate or therapeutic/drug molecule terminals. The modification may comprise oxidation reaction(s) to convert a hydroxy group in the carbohydrate to carboxylic acid.

In various embodiments, p 1. In various embodiments, p is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150. In various embodiments, q≥1. In various embodiments, q is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150.

In various embodiments, the total molecular weight of general formula (I) is kept to no more than about 15,000 or no more than about 10,000. It will be appreciated that copolymerisation may become inefficient when the total molecular weight of general formula (I) and (II) is too high. In various embodiments, when the bioactive PLGA copolymer is used for applications which require fast biodegradation, the molecular weight of general formula (I) is kept low by adjusting the value of p and/or q.

In various embodiments, the repeating unit represented by general formula (II) is in an amount of from about 1 molar % to about 100 molar %, from about 2 molar % to about 99 molar %, from about 3 molar % to about 98 molar %, from about 4 molar % to about 97 molar %, from about 5 molar % to about 96 molar %, from about 10 molar % to about 95 molar %, from about 15 molar % to about 90 molar %, from about 20 molar % to about 85 molar %, from about 25 molar % to about 80 molar %, from about 30 molar % to about 75 molar %, from about 35 molar % to about 70 molar %, from about 40 molar % to about 65 molar %, from about 45 molar % to about 60 molar %, or from about 50 molar % to about 55 molar % relative to the copolymer. In various embodiments, the repeating unit represented by general formula (II) is in an amount of from about 1 molar % to about 10 molar % relative to the copolymer. In various embodiments, the bioactive moiety is about 2 molar %, about 3 molar %, about 4 molar %, about 5 molar %, about 6 molar %, about 7 molar %, about 8 molar %, about 9 molar % or about 10 molar % of the bioactive PLGA copolymer.

In various embodiments, the bioactive PLGA copolymer has a number average molecular weight (Mn) of from about 1,000 to about 300,000, 2,000 to about 250,000, from about 3,000 to about 200,000, from about 4,000 to about 150,000, from about 5,000 to about 100,000, from about 10,000 to about 90,000, from about 20,000 to about 80,000, from about 30,000 to about 70,000, from about 40,000 to about 60,000, or about 50,000.

In various embodiments, the bioactive PLGA copolymer has a polydispersity index (PDI) of from about 1.0 to about 10.0. In various embodiments, PDI of the bioactive PLGA copolymer is about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In various embodiments, the bioactive PLGA copolymer has a polydispersity index (PDI) of from about 1.0 to about 3.0, from about 1.05 to about 2.95, from about 1.1 to about 2.9, from about 1.2 to about 2.8, from about 1.4 to about 2.6, from about 1.6 to about 2.4, from about 1.8 to about 2.2 or about 2.0. In various embodiments, the PDI of the bioactive PLGA copolymer is no more than 1.50.

In various embodiments, the one or more repeating units represented by general formula (I) and the one or more repeating units represented by general formula (II) are designed to link to the poly(norbornene) backbone via at least covalent interactions. In various embodiments, each repeating unit represented by general formula (I) is covalently bonded to the poly(norbornene) backbone and/or each repeating unit represented by general formula (II) is covalently bonded to the poly(norbornene) backbone. Advantageously, as bioactive moieties (in general formula (II)) are covalently bonded to the bioactive PLGA copolymer, bioactivity is localized. In various embodiments, the bioactive moieties do not leach out from the polymer, therefore preventing undesirable/unwanted side effects caused by biomolecules entering the circulatory system and/or reaching unintended parts of the body system. Embodiments of the bioactive synthetic copolymer therefore overcome problems faced by conventional biomolecules that are administered as drugs which may metabolized prematurely before therapeutic effects are achieved. In various embodiments, the bioactive moieties such as drug molecules do not leach out into media which can escape into the environment in the event that disposal is improperly managed.

It will be appreciated that other interactions such as Van der Waals interactions may also be present within the copolymer.

In various embodiments, the bioactive PLGA copolymer comprises a brush, bottlebrush, block, comb or graft-copolymer structure. In various embodiments, the repeating units may be randomly distributed/arranged within the polymer.

In various embodiments, the one or more repeating units represented by general formula (I) comprises two or more different types of bioactive moiety X. In various embodiments, the one or more repeating units represented by general formula (I) comprises 2, 3, 4, 5, 6, 7 or 8 different types of bioactive moiety X. For example, within a bioactive PLGA copolymer, there may be repeating units represented by general formula (I) comprising peptide as X and repeating units represented by general formula (I) comprising carbohydrate as X. Advantageously, in various embodiments, the bioactive PLGA copolymer imparts two or more different types of bioactivities.

In various embodiments, the bioactive PLGA copolymer is a random polymer or a block copolymer. In some embodiments, the block polymer is a di-block or a triblock polymer. For example, the copolymer may have or is made up of two or three different polymer blocks. In some embodiments, the multi-block copolymer comprises more than three polymeric blocks. The blocks may be randomly distributed/arranged within the polymer.

In various embodiments, the bioactive PLGA copolymer is selected from one of the following: PLGA-RGD copolymer comprising RGD in general formula (II) (Scheme 1); and PLGA-HA copolymer comprising HA in general formula (II) (Scheme 2).

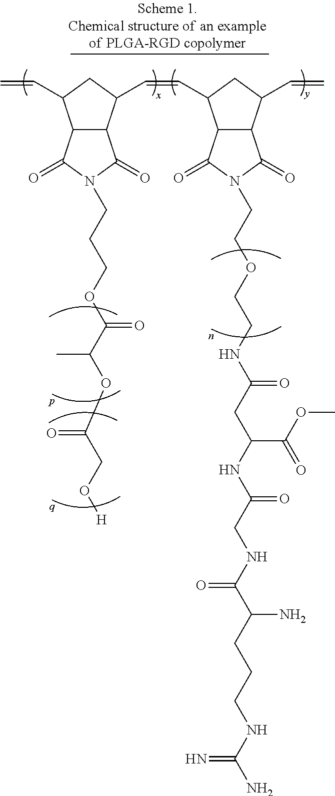

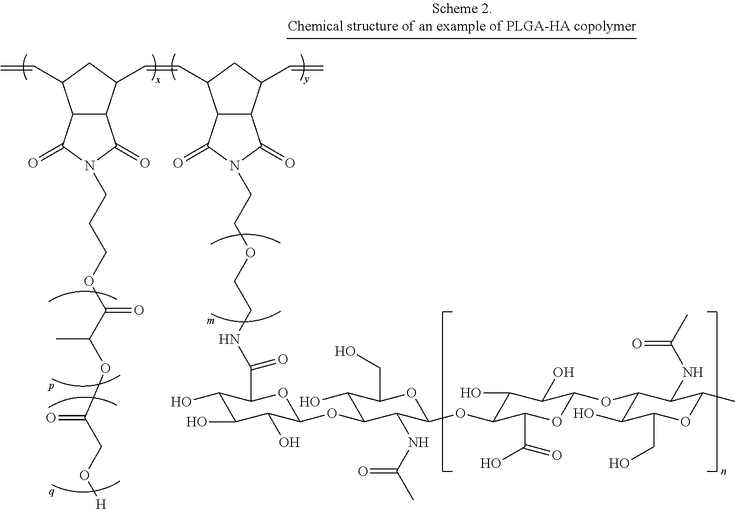

In various embodiments, x≥1. In various embodiments, x is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140,141, 142, 143, 144, 145, 146, 147, 148, 149 or 150. In various embodiments, y≥1. In various embodiments, y is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150. In various embodiments, n≥1. In various embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150.

Advantageously, the bioactive PLGA copolymer disclosed herein is highly customizable. Depending on the application that the bioactive PLGA copolymer is intended, X with the desired biological activity may be selected to match the synthetic polymer PLGA, to eventually obtain the bioactive PLGA copolymer with the desired repeating units represented by general formulae (I) and (II).

It will be appreciated that the bioactive PLGA copolymer disclosed herein may be obtained by ROMP such as that disclosed in PCT application no. PCT/SG2020/050621, which is fully incorporated in its entirety by reference.

### Method of Preparing Bioactive PLGA Material

There is provided a method of preparing a bioactive PLGA material/printed structure or part as disclosed herein, the method comprising:
(i) producing a bioactive PLGA filament; and
(ii) printing a bioactive PLGA material from the bioactive PLGA filament according to a design model to obtain a printed structure or part with pores/channels/micropores/microchannels patterned thereon.

In various embodiments, there is also provided a bioactive polymer filament obtained from the filament production method disclosed herein. Advantageously, the bioactive polymer filament is a feedstock designed for use in fused filament fabrication (FFF) or fused deposition modelling (FDM) method of manufacturing which consists of thermally stable biological molecules that improves bioactivity with adequate mechanical properties. The bioactive polymer filament may comprise/consist essentially of/consist of the base polymer and bioactive copolymer disclosed herein. In various embodiments, the bioactive polymer filament is substantially free from or devoid of other additives such as lubricants.

In various embodiments, the bioactive polymer filament is substantially free from or devoid of other non-medically approved ingredients. In various embodiments, the bioactive polymer filament is a monofilament.

In various embodiments, the step (i) of producing a bioactive PLGA filament comprises:
(i-a) providing a base PLGA powder and a bioactive PLGA copolymer;
(i-b) mixing/blending the base PLGA powder with the bioactive PLGA copolymer to obtain a bioactive PLGA formulation/mixture/blend; and
(i-c) extruding a bioactive PLGA filament from the formulation/mixture/blend.

In various embodiments, the step (ii) of printing a bioactive PLGA material from the bioactive PLGA filament comprises:
(ii-a) feeding the bioactive PLGA filament into a printing apparatus;
(ii-b) applying heat to the bioactive PLGA filament to obtain a molten form of the bioactive PLGA; and
(ii-c) depositing the molten bioactive PLGA on a print bed to form a printed part or structure.

In various embodiments, the printing apparatus comprises a three-dimensional (3D) printing apparatus (e.g., FFF-based or FDM-based 3D printing apparatus). In various embodiments, the printed part or structure comprises a printed three-dimensional part or structure.

Accordingly, there is also provided a fused filament fabrication (FFF) or fused deposition modelling (FDM) based three-dimensional printing method using the bioactive polymer filament disclosed herein as a feedstock. In various embodiments, the method comprises feeding a bioactive polymer filament disclosed herein into a FFF or FDM based three-dimensional printing apparatus (e.g. fed to the print head of the apparatus); applying heat to bioactive polymer filament to obtain a molten/melted form of the bioactive polymer; and depositing the molten/melted bioactive polymer on a print bed to form a printed three-dimensional part or structure. Advantageously, FFF or FDM 3D printing/printers have great advantages such as low cost, shortened time to market and part customisation which are significantly beneficial for medical technology.

In various embodiments, the method further comprises, prior to the step (ii) of printing, digitally modelling/designing a geometry/structure (e.g., 3D geometry/structure) using a digital software. In various embodiments, the FFF or FDM based three-dimensional printing is according to a design model to obtain a structure with the desired design. The bioactive PLGA material may be printed in any shape and size as desired in order to suit a particular application.

In various embodiments, each pore is digitally modelled/designed to have a polygonal shape, polygonal-shaped pore structure or polygonal morphology such as a triangle-shaped, square-shaped, quadrilateral-shaped or hexagonal-shaped pore structure or polygonal morphology. In various embodiments, each pore is digitally modelled/designed to have a polygonal shape having n sides where n≥3 such as a triangular shape (i.e. n=3), square shape (i.e. n=4), quadrilateral shape (i.e. n=4), pentagonal shape (i.e. n=5), hexagonal shape (i.e. n=6), heptagonal shape (i.e. n=7), octagonal shape (i.e. n=8), nonagonal shape (i.e. n=9) or decagonal shape (i.e. n=10).

In various embodiments, each pore is digitally modelled/designed to have a minimum circumscribed circle diameter of about 100.0 μm. In various embodiments, each pore has a circumscribed circle diameter of at least about 100.0 μm, at least about 105.0 μm, at least about 110.0 μm, at least about 115.0 μm, at least about 120.0 μm, at least about 125.0 μm, at least about 130.0 μm, at least about 135.0 μm, at least about 140.0 μm, at least about 145.0 μm, at least about 150.0 μm, at least about 155.0 μm, at least about 160.0 μm, at least about 165.0 μm, at least about 170.0 μm, at least about 175.0 μm, at least about 180.0 μm, at least about 185.0 μm, at least about 190.0 μm, at least about 195.0 μm, at least about 200.0 μm, at least about 205.0 μm, at least about 210.0 μm, at least about 215.0 μm, at least about 220.0 μm, at least about 225.0 μm, at least about 230.0 μm, at least about 235.0 μm, at least about 240.0 μm, at least about 245.0 μm, at least about 250.0 μm, at least about 255.0 μm, at least about 260.0 μm, at least about 265.0 μm, at least about 270.0 μm, at least about 275.0 μm, at least about 280.0 μm, at least about 285.0 μm, at least about 290.0 μm, at least about 295.0 μm, or at least about 300.0 μm. It will be appreciated that as the pore size is governed by design requirements (e.g., the pore size may be customised/adjusted to suit a particular application/requirement), there is no limit to the pore size (e.g., circumscribed circle diameter of the pore).

In various embodiments, each pore is digitally modelled/designed to be uniformly spaced apart from another pore. In various embodiments, each pore is digitally modelled/designed to have a distance that is from about 100.0 μm to about 300.0 μm, from about 105.0 μm to about 295.0 μm, from about 110.0 μm to about 290.0 μm, from about 115.0 μm to about 285.0 μm, from about 120.0 μm to about 280.0 μm, from about 125.0 μm to about 275.0 μm, from about 130.0 μm to about 270.0 μm, from about 135.0 μm to about 265.0 μm, from about 140.0 μm to about 260.0 μm, from about 145.0 μm to about 255.0 μm, from about 150.0 μm to about 250.0 μm, from about 155.0 μm to about 245.0 μm, from about 160.0 μm to about 240.0 μm, from about 165.0 μm to about 235.0 μm, from about 170.0 μm to about 230.0 μm, from about 175.0 μm to about 225.0 μm, from about 180.0 μm to about 220.0 μm, from about 185.0 μm to about 215.0 μm, from about 190.0 μm to about 210.0 μm, from about 195.0 μm to about 205.0 μm, or about 200.0 μm from another pore.

In various embodiments, the pores are digitally modelled/designed to be surrounded by a buffer zone/area.

In various embodiments, the buffer zone/area is digitally modelled/designed to form the border of the bioactive PLGA material/printed structure or part.

In various embodiments, the buffer zone/area is digitally modelled/designed to have a width that is from about 1 mm to about 10 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, or about 10.0 mm.

In various embodiments, the bioactive PLGA material is printed with a thickness/height/depth of from about 0.05 mm to about 0.60 mm, from about 0.06 mm to about 0.59 mm, from about 0.07 mm to about 0.58 mm, from about 0.08 mm to about 0.57 mm, from about 0.09 mm to about 0.56 mm, from about 0.10 mm to about 0.55 mm, from about 0.11 mm to about 0.54 mm, from about 0.12 mm to about 0.53 mm, from about 0.13 mm to about 0.52 mm, from about 0.14 mm to about 0.51 mm, from about 0.15 mm to about 0.50 mm, from about 0.16 mm to about 0.49 mm, from about 0.17 mm to about 0.48 mm, from about 0.18 mm to about 0.47 mm, from about 0.19 mm to about 0.46 mm, from about 0.20 mm to about 0.45 mm, from about 0.21 mm to about 0.44 mm, from about 0.22 mm to about 0.43 mm, from about 0.23 mm to about 0.42 mm, from about 0.24 mm to about 0.41 mm, from about 0.25 mm to about 0.40 mm, from about 0.26 mm to about 0.39 mm, from about 0.27 mm to about 0.38 mm, from about 0.28 mm to about 0.37 mm, from about 0.29 mm to about 0.36 mm, from about 0.30 mm to about 0.35 mm, from about 0.31 mm to about 0.34 mm, or from about 0.32 mm to about 0.33 mm.

In various embodiments, the step of applying heat is at a temperature that is based on a predetermined melt/softening temperature and a predetermined onset degradation temperature of the bioactive PLGA. For example, the printing may be performed at a temperature (e.g. the temperature of the print head) that is no more than the temperature at which the bioactive moiety part of the filament degrades/decomposes/disintegrates/depolymerises/breaks down. In various embodiments, the printing is performed at a temperature that is between the melt/softening temperature of bioactive PLGA filament and the onset degradation temperature of the bioactive PLGA. In various embodiments, the printing temperature is dependent on the bioactive PLGA filament used for 3D printing.

In various embodiments, the step of printing is performed at a temperature that is from about 15° C. to about 40° C. above the melting/softening point of the bioactive PLGA filament and up to 5° C. below the degradation point of the bioactive PLGA. In some embodiments, the printing is performed at a temperature that is from about 15° C. to about 40° C., from about 16° C. to about 39° C., from about 17° C. to about 38° C., from about 18° C. to about 37° C., from about 19° C. to about 36° C., from about 20° C. to about 35° C., from about 21° C. to about 34° C., from about 22° C. to about 33° C., from about 23° C. to about 32° C., from about 24° C. to about 31° C., from about 25° C. to about 30° C., from about 26° C. to about 29° C., or from about 27° C. to about 28° C. above the melting/softening point/temperature of the bioactive polymer filament. In some embodiments, the printing is performed at a temperature that is no more than about 5° C., no more than about 4.5° C., no more than about 4° C., no more than about 3.5° C., no more than about 3° C., no more than about 2.5° C., no more than about 2° C., no more than about 1.5° C., no more than about 1° C., no more than about 0.5° C., no more than about 0.4° C., no more than about 0.3° C., no more than about 0.2° C., or no more than about 0.1° C. below the degradation point/temperature of the bioactive PLGA.

In various embodiments, the step of printing is performed in the presence of a base plate/build plate/print bed that has a temperature that is no less than the temperature at which the thermoplastic solidifies or converts into solid state. In various embodiments, the base plate temperature is dependent on the bioactive polymer filament used for 3D printing. The temperature of the base plate may range from room temperature (e.g., no heating) and up to about 15° C. above the melting/softening temperature of the bioactive PLGA filament. In some embodiments, the printing is performed in the presence of a base plate having a temperature that is no more than about 15° C., no more than about 14° C., no more than about 13° C., no more than about 12° C., no more than about 11° C., no more than about 10° C., no more than about 9° C., no more than about 8° C., no more than about 7° C., no more than about 6° C., no more than about 5° C., no more than about 4° C., no more than about 3° C., no more than about 2° C., or no more than about 1° C. above the melting/softening point/temperature of the bioactive PLGA filament.

In various embodiments, the step of printing is performed in the presence of a base plate/build plate/print bed having a temperature ranging from about 25° C. to about 80° C., from about 30° C. to about 75° C., from about 35° C. to about 70° C., from about 36° C. to about 69° C., from about 37° C. to about 68° C., from about 38° C. to about 67° C., from about 39° C. to about 66° C., from about 40° C. to about 65° C., from about 41° C. to about 64° C., from about 42° C. to about 63° C., from about 43° C. to about 62° C., from about 44° C. to about 61° C., from about 45° C. to about 60° C., from about 46° C. to about 59° C., from about 47° C. to about 58° C., from about 48° C. to about 57° C., from about 49° C. to about 56° C., from about 50° C. to about 55° C., from about 51° C. to about 54° C., from about 52° C. to about 53° C., or about 52.5° C.

In various embodiments, the step of printing is performed at a temperature ranging from about 110° C. to about 160° C., from about 115° C. to about 155° C., from about 120° C. to about 150° C., from about 125° C. to about 145° C., from about 130° C. to about 140° C., from about 131° C. to about 139° C., from about 132° C. to about 138° C., from about 133° C. to about 137° C., from about 134° C. to about 136° C., or about 135° C.

In various embodiments, the bioactive PLGA material is printed with a printing nozzle having a diameter of from about 0.10 mm to about 1.00 mm, from about 0.11 mm to about 0.99 mm, from about 0.12 mm to about 0.98 mm, from about 0.13 mm to about 0.97 mm, from about 0.14 mm to about 0.96 mm, from about 0.15 mm to about 0.95 mm, from about 0.20 mm to about 0.90 mm, from about 0.25 mm to about 0.85 mm, from about 0.30 mm to about 0.80 mm, from about 0.35 mm to about 0.75 mm, from about 0.40 mm to about 0.70 mm, from about 0.45 mm to about 0.65 mm, from about 0.50 mm to about 0.60 mm, from about 0.51 mm to about 0.59 mm, from about 0.52 mm to about 0.58 mm, from about 0.53 mm to about 0.57 mm, from about 0.54 mm to about 0.56 mm, or about 0.55 mm.

In various embodiments, the step of printing is performed at a printing speed of from about 1.0 mm/s to about 70.0 mm/s, from about 2.0 mm/s to about 69.0 mm/s, from about 3.0 mm/s to about 68.0 mm/s, from about 4.0 mm/s to about 67.0 mm/s, from about 5.0 mm/s to about 66.0 mm/s, from about 6.0 mm/s to about 65.0 mm/s, from about 7.0 mm/s to about 64.0 mm/s, from about 8.0 mm/s to about 63.0 mm/s, from about 9.0 mm/s to about 62.0 mm/s, from about 10.0 mm/s to about 61.0 mm/s, from about 15.0 mm/s to about 60.0 mm/s, from about 20.0 mm/s to about 55.0 mm/s, from about 25.0 mm/s to about 50.0 mm/s, from about 30.0 mm/s to about 45.0 mm/s, or from about 35.0 mm/s to about 40.0 mm/s.

In various embodiments, the bioactive PLGA material is printed with an infill density of from about 70.0% to about 100.0%, from about 71.0% to about 99.9%, from about 72.0% to about 99.8%, from about 73.0% to about 99.7%, from about 74.0% to about 99.6%, from about 75.0% to about 99.5%, from about 76.0% to about 99.0%, from about 77.0% to about 98.0%, from about 78.0% to about 97.0%, from about 79.0% to about 96.0%, from about 80.0% to about 95.0%, from about 81.0% to about 94.0%, from about 82.0% to about 93.0%, from about 83.0% to about 92.0%, from about 84.0% to about 91.0%, from about 85.0% to about 90.0%, from about 86.0% to about 89.0%, or from about 87.0% to about 88.0%.

In various embodiments, the bioactive PLGA material is printed with an infill overlap of from about 0.01 mm to about 0.20 mm, from about 0.02 mm to about 0.19 mm, from about 0.03 mm to about 0.18 mm, from about 0.04 mm to about 0.17 mm, from about 0.05 mm to about 0.16 mm, from about 0.06 mm to about 0.15 mm, from about 0.07 mm to about 0.14 mm, from about 0.08 mm to about 0.13 mm, from about 0.09 mm to about 0.12 mm, or from about 0.10 mm to about 0.11 mm.

In various embodiments, the bioactive PLGA material is printed with a layer height of from about 0.01 mm to about 0.15 mm, from about 0.02 mm to about 0.14 mm, from about 0.03 mm to about 0.13 mm, from about 0.04 mm to about 0.12 mm, from about 0.05 mm to about 0.11 mm, from about 0.06 mm to about 0.10 mm, from about 0.07 mm to about 0.09 mm, or about 0.08 mm.

In various embodiments, the bioactive PLGA material is printed with a line width of from about 0.10 mm to about 0.50 mm, from about 0.11 mm to about 0.49 mm, from about 0.12 mm to about 0.48 mm, from about 0.13 mm to about 0.47 mm, from about 0.14 mm to about 0.46 mm, from about 0.15 mm to about 0.45 mm, from about 0.16 mm to about 0.44 mm, from about 0.17 mm to about 0.43 mm, from about 0.18 mm to about 0.42 mm, from about 0.19 mm to about 0.41 mm, from about 0.20 mm to about 0.40 mm, from about 0.21 mm to about 0.39 mm, from about 0.22 mm to about 0.38 mm, from about 0.23 mm to about 0.37 mm, from about 0.24 mm to about 0.36 mm, from about 0.25 mm to about 0.35 mm, from about 0.26 mm to about 0.34 mm, from about 0.27 mm to about 0.33 mm, from about 0.28 mm to about 0.32 mm, from about 0.29 mm to about 0.31 mm, or about 0.30 mm.

In various embodiments, the printing apparatus (e.g., FFF-based or FDM-based three-dimensional printing apparatus) is configured for filament feedstock having filament diameters falling in the range of from about 1.5 mm to about 4.0 mm, from about 1.6 mm to about 3.9 mm, from about 1.7 mm to about 3.5 mm, from about 1.71 mm to about 3.4 mm, from about 1.72 mm to about 3.3 mm, from about 1.73 mm to about 3.2 mm, from about 1.74 mm to about 3.1 mm, from about 1.75 mm to about 3.0 mm, from about 1.76 mm to about 2.95 mm, from about 1.77 mm to about 2.90 mm, from about 1.78 mm to about 2.88 mm, from about 1.79 mm to about 2.86 mm, or from about 1.80 mm to about 2.85 mm.

Advantageously, in various embodiments, the printing method and bioactive polymer filament feedstock is capable of being used by commercially available FFF or FDM based three-dimensional printers or extruders.

In various embodiments, the bioactive PLGA copolymer is present in powdered form. Accordingly, it will be appreciated that in these embodiments, no additional/further step may be required/necessary to convert the bioactive PLGA copolymer into powdered form.

In various embodiments, the step (i-c) of extruding a bioactive PLGA filament from the formulation/mixture/blend is performed at an extrusion temperature profile that is based on a predetermined melt/softening temperature and a predetermined onset degradation temperature of the bioactive PLGA. In various embodiments, the extrusion temperature profile is based on a predetermined melt/softening temperature of the base polymer or the bioactive polymer, whichever is higher. For example, if the melt temperature of the base polymer is higher than the melt temperature of the bioactive polymer, then the extrusion temperature profile is based on the predetermined melt/softening temperature of the base polymer, and vice versa.

In various embodiments, the bioactive polymer filament is suitable for use as a feedstock for fused filament fabrication (FFF) or fused deposition modelling (FDM) based 3D printing. FFF is an extrusion-based 3D printing technology which generally utilizes polymer filament feedstock. It deposits the molten polymer in 2-dimensional plane according to the deposition path set by the machine over several layers until the part is fully printed in 3-dimensional space. FFF technology typically uses filaments made of thermoplastic polymers where heat transfer characteristics and rheology are important properties required for good quality printed parts. FFF is preferred over other 3D printing technology as it enables good control over printing parameters which can influence the mechanical properties. However, due to high temperature utilized in FFF technology, living cells or other temperature-sensitive biological molecules may not be incorporated in its filament as it would denature the proteins, degrade polysaccharides or oligopeptides and kill the cells. Advantageously, various embodiments of the bioactive polymer filament disclosed herein provide desirable mechanical as well as biological characteristics that makes its suitable for use in printing medical-related structures using FFF 3D printing technologies.

In various embodiments, the bioactive polymer filament and/or bioactive copolymer is acellular or is substantially devoid of cells. Advantageously, in various embodiments, the bioactive polymer filament and/or bioactive copolymer do not rely on highly temperature sensitive biological moieties like stem cells or growth factors to impart bioactivity since these biological moieties are highly susceptible to cell death during the extrusion process to form the filament feedstock. Even more advantageously, embodiments of the bioactive polymer filament and/or bioactive copolymer are still able to stimulate host cells to proliferate which promotes tissue growth.

In various embodiments, the bioactive copolymer comprises biological molecules or biomolecules that are bonded/linked (e.g., chemically bonded/linked) to/on the bioactive copolymer. It will be appreciated that in various embodiments, the bioactive polymer filament and/or bioactive copolymer is substantially devoid of free (or unbound/unbonded/unlinked) biomolecules such as free (or unbound/unbonded/unlinked) oligopeptides or oligosaccharides.

In various embodiments, the method further comprises, prior to the step (ii) of printing, performing a post-extrusion thermal analysis on the extruded bioactive PLGA filament to assess onset degradation of the bioactive PLGA in the filament.

In various embodiments, the method further comprises performing one or more of post-printing analysis of the printed three-dimensional part or structure, the post-printing analysis selected from the group consisting of:
i. a mechanical analysis of the printed three-dimensional part or structure e.g. to assess its mechanical properties;
ii. a biocompatibility analysis of the printed three-dimensional part or structure e.g. to assess its biocompatibility with living cells;
iii. a thermal analysis on the printed three-dimensional part or structure e.g. to assess onset degradation of the bioactive polymer in the printed three-dimensional part or structure; and
iv. a spectrometric analysis of the printed three-dimensional part or structure to assess the presence of bioactive copolymer in the printed three-dimensional part or structure. For example, nuclear magnetic resonance (NMR) spectroscopy may be performed on the filament and/or the printed three-dimensional part or structure.

The mechanical analysis may be performed under ASTM standards or other equivalent standards to determine the properties of the printed structure and whether it is suitable for its specific use. It will be appreciated that any other test methods that are equivalent to the ASTM standards may be used as well. Furthermore, the biocompatibility tests may be carried out with various human cell lines which the materials are designed to interact with. The thermal analysis may comprise one or more of thermogravimetric analysis (TGA) and differential scanning calorimetry (DSC). In various embodiments, the thermal analysis comprises simultaneous thermal analysis (STA) through the simultaneous application of TGA and DSC.

In various embodiments, the base PLGA powder is obtained from physical processes to reduce the size of base polymer pellets. Such physical processes may comprise grinding, pulverizing, milling, cryogenic milling/cryomilling or combinations thereof to obtain the powdered base polymer. Accordingly, in various embodiments, the method may further comprise performing one or more of grinding, pulverizing, milling, cryogenic milling/cryomilling of base polymer pellets to obtain base polymer powder. In one example, the base polymer powder is obtained from cryogenic milling/cryomilling of the base polymer pellets. The cryomiling/cryogenic milling may be performed in the presence of a cryogenic liquid selected from the group consisting of argon, helium, hydrogen, nitrogen and oxygen. Advantageously, using cryomilling/cryogenic milling/grinding in embodiments of the method disclosed herein aids in embrittlement process and/or prevents degradation of the polymer. It will be appreciated that the cryogen/cryogenic liquid used (e.g., liquid nitrogen) can lower the temperature significantly (<−196° C.) which in turn may induce embrittlement on the polymer/material, and subsequently easing the milling process. It will also be appreciated that the cryogen/cryogenic liquid used can prevent any thermal degradation of the polymer/material from occurring during the high-energy milling process.

In various embodiments, the base PLGA powder has an average particle size of no more than about 1 mm, from about 0.50 mm to about 1 mm, from about 0.55 mm to about 0.95 mm, from about 0.50 mm to about 0.90 mm, from about 0.50 mm to about 0.85 mm, from about 0.50 mm to about 0.80 mm, from about 0.60 mm to about 1 mm, from about 0.65 mm to about 1 mm, or from about 0.70 mm to about 0.95 mm. In various embodiments, the base PLGA powder has an average particle size of from about 0.10 mm to about 1.00 mm, from about 0.11 mm to about 0.99 mm, from about 0.12 mm to about 0.98 mm, from about 0.13 mm to about 0.97 mm, from about 0.14 mm to about 0.96 mm, from about 0.15 mm to about 0.95 mm, from about 0.20 mm to about 0.90 mm, from about 0.25 mm to about 0.85 mm, from about 0.30 mm to about 0.80 mm, from about 0.35 mm to about 0.75 mm, from about 0.40 mm to about 0.70 mm, from about 0.45 mm to about 0.65 mm, from about 0.50 mm to about 0.60 mm, from about 0.51 mm to about 0.59 mm, from about 0.52 mm to about 0.58 mm, from about 0.53 mm to about 0.57 mm, from about 0.54 mm to about 0.56 mm, or about 0.55 mm.

In various embodiments, the base PLGA and/or bioactive PLGA copolymer have been dried (e.g. vacuum dried) prior to mixing. Accordingly, in various embodiments, the method further comprises drying (e.g. vacuum drying) the base PLGA and/or bioactive PLGA copolymer prior to mixing them. For example, after cryomiling/cryogenic milling is carried out on base polymer pellets to obtain base polymer powder, the base polymer powder is dried (e.g. vacuum dried) prior to mixing with the bioactive copolymer. Advantageously, vacuum drying provides an inert environment whereby moisture may be reduced significantly allowing for more effective drying. Additionally, since airflow is absent in vacuum drying, materials in powder form are not blown around which would otherwise result in material loss. The drying step may be performed at an ambient or room temperature or at temperature of from about 35° C. to about 100° C., from about 40° C. to about 95° C., from about 45° C. to about 90° C., from about 40° C. to about 85° C. or from about 45° C. to about 80° C. The drying step may be performed over a time period of about 4 hours, about 5 hours, about 6 hours, about 7 hours, about 8 hours, about 9 hours, about 10 hours, about 11 hours, about 12 hours, from about 4 hours to about 12 hours, about 24 hours, about 36 hours, or about 48 hours. In various embodiments, the drying step comprises vacuum drying at a drying pressure of from about −50 mmHg to about −10 mmHg, from about −40 mmHg to about −20 mmHg, or about −40 mmHg.

In various embodiments, the method further comprises performing a pre-extrusion thermal analysis on the bioactive copolymer and/or base polymer to determine the melt temperature and the onset degradation temperature of the bioactive polymer/copolymer and/or base polymer.

The method may further comprise performing thermal analysis on base polymer pellets prior to reducing their sizes (e.g. cryogenic milling/cryomilling) to obtain powdered forms and optionally performing thermal analysis on base polymer pellets after the powdered forms are obtained (e.g. cryogenic milling/cryomilling). Advantageously, the thermal analysis may be useful to determine the thermal properties of the base polymer prior to size reduction (e.g. cryogenic milling/cryomilling) and/or after size reduction and/or prior to filament extrusion so that a benchmark may be obtained and an assessment may be made on whether there is a detraction in the physical and/or thermal properties of the subsequently obtained filament feedstock or printed structure from that expected (e.g. based on the original mechanical properties of the base polymer). The thermal analysis may also provide useful information on the melting temperature (if any) and/or degradation temperature of the base polymer (e.g. pellet form or powdered form) so that the extrusion temperature profile may be customised for the particular base polymer e.g. based on the melt temperature and the onset degradation temperature of the base polymer that were determined. For instance, the extrusion temperature profile may be customised such that the extrusion temperature is between the melting/softening temperature and degradation temperature of the base polymer. It will also be appreciated that in some embodiments, the determination of the melting/softening temperature and degradation temperature of the base polymer may have already been completed previously or such information are already readily available for known/established polymers. Thus, in such embodiments, it may be optional for the presently disclosed method to have such active determination steps.

Similarly, the method may further comprise performing thermal analysis on bioactive copolymer prior to mixing with the base polymer and/or prior to filament extrusion. Advantageously, the thermal analysis may be useful to determine the thermal properties of the bioactive copolymer prior to mixing with the base polymer and/or prior to filament extrusion so that a benchmark may be obtained and an assessment may be made on whether there is a detraction in the physical and/or thermal properties of the subsequently obtained filament feedstock or printed structure that expected (e.g. based on the properties of the bioactive copolymer prior to extrusion). The thermal analysis may also provide useful information on the melting temperature and/or degradation temperature of the bioactive polymer/copolymer so that the extrusion temperature profile may be customised for the particular bioactive copolymer e.g. based on the melt temperature and the onset degradation temperature of the base polymer that were determined. For instance, the extrusion temperature profile may be customised such that the extrusion temperature is between the melting/softening temperature and degradation temperature of the bioactive polymer/copolymer. In various embodiments, such active determination steps are present in the method disclosed herein. Advantageously, adopting/employing such active determination steps in the method disclosed herein prevents degradation of the bioactive polymer/copolymer during filament extrusion and preserves bioactivity of the bioactive copolymer/bioadditive in the filament. As biomolecules linked/bonded to/on the polymer may be lost/melted/degraded during heat treatment, it may therefore be important to determine the temperature(s) at which such situation(s) may occur. It will also be appreciated that in some embodiments, the determination of the melting/softening temperature and degradation temperature of the bioactive polymer/copolymer may have already been completed previously or such information are already readily available for such polymers. Thus, in such embodiments, it may be optional for the presently disclosed method to have such active determination steps.

In various embodiments, the extrusion temperature profile is based on predetermined melt/softening temperatures and predetermined onset degradation temperatures of both the base polymer and the bioactive polymer. Accordingly, the extrusion temperature profile may be customised such that the extrusion temperature is between the melting/softening temperature and onset degradation temperature of the bioactive copolymer and also between the melting/softening temperature and onset degradation temperature of the base polymer (e.g. base polymer powder).

In various embodiments, the thermal analysis disclosed herein comprises one or more of thermogravimetric analysis (TGA) and differential scanning calorimetry (DSC). In various embodiments, the thermal analysis comprises application of TGA and DSC. The application of TGA and DSC may be non-simultaneous or simultaneous. For example, the thermal analysis comprising TGA and DSC may be performed either non-simultaneously (e.g., TGA-DSC) or simultaneously (e.g., STA). In various embodiments, the thermal analysis comprises simultaneous thermal analysis (STA) through the simultaneous application of TGA and DSC. For example, the post-extrusion thermal analysis may comprise STA.

In various embodiments, the extruding is performed using an extruder having one or more rotating screws. The extruder may have one or more, or two or more rotating screws. For example, the extruder may be a single screw extruder. Advantageously, as opposed to a single screw extruder, an extruder with two or more rotating screws may achieve good and uniform mixing/blending of the base polymer and bioactive copolymer. In one example, the extruder is a twin screw extruder (TSE). In another example, the extruder is a multi-screw extruder. The twin screw and/or multi-screw extruder may be intermeshing or non-intermeshing and co-rotating or counter-rotating. Each screw may be made of multiple screw elements to support either feeding, mixing or discharging. In various embodiments, the mixing elements along each screw offers a unique advantage as it kneads the molten blended materials to achieve homogeneity. For example, in various embodiments, these elements form at three different segments along each screw, granting a thorough mixing process.

In various embodiments, the extruder comprises a melt pump for building pressure and for ensuring constant output. The nozzle size of the extruder or melt pump of the extruder may have a diameter that allows for a filament diameter falling in the range of from about 1.5 mm to about 4.0 mm, from about 1.6 mm to about 3.9 mm, from about 1.7 mm to about 3.5 mm, from about 1.71 mm to about 3.4 mm, from about 1.72 mm to about 3.3 mm, from about 1.73 mm to about 3.2 mm, from about 1.74 mm to about 3.1 mm, from about 1.75 mm to about 3.0 mm, from about 1.76 mm to about 2.95 mm, from about 1.77 mm to about 2.90 mm, from about 1.78 mm to about 2.88 mm, from about 1.79 mm to about 2.86 mm, or from about 1.80 mm to about 2.85 mm to be produced. The extruder or melt pump of the extruder may have a nozzle diameter that falls in the range of from about 1 mm to about 4 mm, about 1 mm, about 1.5 mm, about 2 mm, about 2.5 mm, about 3 mm, about 3.5 mm or about 4 mm. The extruder or melt pump of the extruder may have a nozzle diameter that falls in the range of from about 1.00 mm to about 4.00 mm, from about 1.05 mm to about 3.95 mm, from about 1.10 mm to about 3.90 mm, from about 1.15 mm to about 3.85 mm, from about 1.20 mm to about 3.80 mm, from about 1.25 mm to about 3.75 mm, from about 1.30 mm to about 3.70 mm, from about 1.35 mm to about 3.65 mm, from about 1.40 mm to about 3.60 mm, from about 1.45 mm to about 3.55 mm, from about 1.50 mm to about 3.50 mm, from about 1.55 mm to about 3.45 mm, from about 1.60 mm to about 3.40 mm, from about 1.65 mm to about 3.35 mm, from about 1.70 mm to about 3.30 mm, from about 1.75 mm to about 3.25 mm, from about 1.80 mm to about 3.20 mm, from about 1.85 mm to about 3.15 mm, from about 1.90 mm to about 3.10 mm, from about 1.95 mm to about 3.05 mm, from about 2.00 mm to about 3.00 mm, from about 2.05 mm to about 2.95 mm, from about 2.10 mm to about 2.90 mm, from about 2.15 mm to about 2.85 mm, from about 2.20 mm to about 2.80 mm, from about 2.25 mm to about 2.75 mm, from about 2.30 mm to about 2.70 mm, from about 2.35 mm to about 2.65 mm, from about 2.40 mm to about 2.60 mm, from about 2.45 mm to about 2.55 mm, or about 2.50 mm.

In various embodiments, the extruded bioactive polymer filament has a filament diameter falling in the range of from about 1.5 mm to about 4.0 mm, from about 1.6 mm to about 3.9 mm, from about 1.7 mm to about 3.5 mm, from about 1.71 mm to about 3.4 mm, from about 1.72 mm to about 3.3 mm, from about 1.73 mm to about 3.2 mm, from about 1.74 mm to about 3.1 mm, from about 1.75 mm to about 3.0 mm, from about 1.76 mm to about 2.95 mm, from about 1.77 mm to about 2.90 mm, from about 1.78 mm to about 2.88 mm, from about 1.79 mm to about 2.86 mm, or from about 1.80 mm to about 2.85 mm. The extruder may further comprise a water bath and/or haul unit.

In various embodiments, the formulation/mixture of base polymer and bioactive copolymer comprises from about 60.0 wt % to about 99.9 wt %, from about 61.0 wt % to about 99.8 wt %, from about 62.0 wt % to about 99.7 wt %, from about 63.0 wt % to about 99.6 wt %, from about 64.0 wt % to about 99.5 wt %, from about 65.0 wt % to about 99.0 wt %, from about 66.0 wt % to about 98.5 wt %, from about 67.0 wt % to about 98.0 wt %, from about 68.0 wt % to about 97.0 wt %, from about 69.0 wt % to about 96.0 wt %, from about 70.0 wt % to about 95.0 wt %, from about 71.0 wt % to about 94.0 wt %, from about 72.0 wt % to about 93.0 wt %, from about 73.0 wt % to about 92.0 wt %, from about 74.0 wt % to about 91.0 wt %, from about 75.0 wt % to about 90.0 wt %, from about 76.0 wt % to about 89.0 wt %, from about 77.0 wt % to about 88.0 wt %, from about 78.0 wt % to about 87.0 wt %, from about 79.0 wt % to about 86.0 wt %, from about 80.0 wt % to about 85.0 wt %, from about 81.0 wt % to about 84.0 wt %, or from about 82.0 wt % to about 83.0 wt % of the base polymer.

In various embodiments, the formulation/mixture of base polymer and bioactive copolymer comprises from about 0.1 wt % to about 40.0 wt %, from about 0.1 wt % to about 39.0 wt %, from about 0.2 wt % to about 38.0 wt %, from about 0.3 wt % to about 37.0 wt %, from about 0.4 wt % to about 36.0 wt %, from about 0.5 wt % to about 35.0 wt %, from about 1.0 wt % to about 34.0 wt %, from about 1.5 wt % to about 33.0 wt %, from about 2.0 wt % to about 32.0 wt %, from about 3.0 wt % to about 31.0 wt %, from about 4.0 wt % to about 30.0 wt %, from about 5.0 wt % to about 29.0 wt %, from about 6.0 wt % to about 28.0 wt %, from about 7.0 wt % to about 27.0 wt %, from about 8.0 wt % to about 26.0 wt %, from about 9.0 wt % to about 25.0 wt %, from about 10.0 wt % to about 24.0 wt %, from about 11.0 wt % to about 23.0 wt %, from about 12.0 wt % to about 22.0 wt %, from about 13.0 wt % to about 21.0 wt %, from about 14.0 wt % to about 20.0 wt %, from about 15.0 wt % to about 19.0 wt %, from about 16.0 wt % to about 18.0 wt %, or about 17.0 wt % of the bioactive copolymer.

In various embodiments, the material is a non-animal derived material. In various embodiments, the material/printed structure is acellular or is substantially devoid of cells. Advantageously, in various embodiments, the material/printed structure do not rely on highly temperature sensitive biological moieties like stem cells or growth factors to impart bioactivity. Even more advantageously, embodiments of the material/printed structure are still able to stimulate host cells to proliferate which promotes skin and/or tissue growth.

In various embodiments, the material/printed structure is biocompatible, i.e. the material is compatible with biological systems or parts of the biological systems without substantially or significantly eliciting an adverse physiological response such as a toxic reaction/response, an immune reaction/response, an injury or the like when used on the human or animal body. In various embodiments, the material is substantially devoid of substances that elicit an adverse physiological response.

There is also provided a bioactive PLGA material, blend, filament, or printed structure as disclosed herein for use in medicine. The material may also be a material that is suitable for use in stimulating/promoting skin regeneration, tissue regeneration, wound healing, wound regeneration, wound regrowth, wound repair, wound closure and/or wound treatment. The material may also be a material that is suitable for use in controlling/reducing inflammation and/or reducing risk of scarring or scar formation.

There is provided use of a bioactive PLGA material, blend, filament, or printed structure as disclosed herein in the manufacture of a medicament for stimulating/promoting skin regeneration, tissue regeneration, wound healing, wound regeneration, wound regrowth, wound repair, wound closure and/or wound treatment. There is also provided use of a bioactive PLGA material, blend, filament, or printed structure as disclosed herein in the manufacture of a medicament for controlling/reducing inflammation and/or reducing risk of scarring or scar formation.

There is provided a method of stimulating/promoting skin regeneration, tissue regeneration, wound healing, wound regeneration, wound regrowth, wound repair, wound closure and/or wound treatment, the method comprising applying the bioactive PLGA material, blend, filament, or printed structure as disclosed herein to a body of a subject in need thereof. There is also provided a method of controlling/reducing inflammation and/or reducing risk of scarring or scar formation, the method comprising applying the bioactive PLGA material, blend, filament, or printed structure as disclosed herein to a body of a subject in need thereof.

In various embodiments, there is also provided a bioactive PLGA material, blend, filament, or printed structure as disclosed herein for use in the prophylaxis or treatment of wounds. There is also provided a method of treating a wound, the method comprising applying the material, blend, filament, or printed structure disclosed herein to a wound of a subject in need thereof. In various embodiments, the method further comprises evaluating one or more wound characteristic; determining a treatment needed; three-dimensionally printing a bioactive PLGA material that is/are responsive to the treatment needed; and/or administering/applying said material, blend, filament, or printed structure to the wound. There is also provided use of a bioactive PLGA blend/filament/material/printed structure disclosed herein in the manufacture of a medicament for the prophylaxis or treatment of wounds. The wound may be a dermal wound or a skin wound. The wound may also be selected from acute wounds such as burns, incisions, excisions (e.g., superficial, partial thickness, full thickness incisional/excisional wounds etc), chronic and/or slow healing wounds such as pressure sores, venous ulcers, diabetic ulcers, foot ulcers, bed sores, the like or combinations thereof.

In various embodiments, the bioactive PLGA material disclosed herein is different from those of the art in that embodiments of the bioactive PLGA material is 3D printed into dermal templates using PLGA-based materials at different blending ratio. Advantageously, embodiments of the bioactive PLGA material disclosed herein demonstrate promising porcine data e.g., wound healing results, which was designed using relevant test protocols for this application, that are not available/shown and cannot be expected from the art.

In various embodiments, the bioactive PLGA material disclosed herein is completely different from those known in the art that comprises porcine dermal collagen-bonded nylon membrane on a silicone backing (e.g., in a biosynthetic dressing).

In various embodiments, the bioactive PLGA material disclosed herein is different from those known in the art at least in the fabrication method and materials used. For example, various embodiments of the bioactive PLGA material disclosed herein are different from those of the art that utilizes PCL-collagen and electrospinning process. In contrast, various embodiments of the bioactive PLGA material disclosed herein relates to fused filament fabrication (FFF) 3D printing with bioactive PLGA copolymers (e.g., PLGA-RGD and PLGA-HA copolymers). Various embodiments of the bioactive PLGA material disclosed herein are also different from scaffolds known in the art that requires an additional step in obtaining its micropores such as manually creating the micropores via acupuncture needle or Derma stamp.

In various embodiments, the bioactive PLGA material disclosed herein is different from those of the art at least in the materials used, fabrication method and objective. For example, various embodiments of the bioactive PLGA material disclosed herein are different from those of the art that focuses on the role of water vapor transmission rates (WVTR) in wound healing using PU membranes with varying porosities through a particulate leaching method using a solution consisting of polyurethane (PU), sodium citrate and dimethylformamide (DMF) at different ratios. In contrast, various embodiments of the bioactive PLGA material disclosed herein relates to utilizing bioactive PLGA copolymers (e.g., PLGA-RGD and PLGA-HA) with FFF 3D printing in creating dermal templates with micropores. Various embodiments of the bioactive PLGA material disclosed herein are completely different from those of the art that do not contain any bioactive materials.

### BRIEF DESCRIPTION OF FIGURES

FIG. 1 shows a bioactive poly(lactic-co-glycolic acid) (PLGA) material 100 designed in accordance with various embodiments disclosed herein. The bioactive PLGA material 100 comprises pores 102 patterned on said material. The bioactive PLGA material 100 also comprises a base PLGA and a bioactive PLGA copolymer. The bioactive PLGA copolymer comprises a polymer backbone 104 (e.g., poly(norbornene dicarboximide) backbone), pendant arms of PLGA 106a, 106b and 106c, and pendant arms of biomolecules/bioactive molecules 108a, 108b and 108c tethered on hydrophilic linker (e.g., PEG chains 110a, 110b and 110c). As shown in the schematic diagram, the pendant arms are attached to the poly(norbornene dicarboximide) backbone 104. 108a, 108b and 108c may be the same or different types of biomolecules or bioactive moieties. In various embodiments, the 3D printed dermal scaffold comprises pores for fluid release and bioactive polymer additive where the biomolecule used is, for example, RGD peptide or hyaluronic acid of 8-13 saccharide units.

FIG. 2 shows images obtained from porcine burn wound healing studies of negative control, sham (no treatment) in accordance with an embodiment disclosed herein. Images were captured on Day 3, Day 7, Day 15 and Day 21.

FIG. 3 shows images obtained from porcine burn wound healing studies of 5% PLGA-RGD in accordance with an embodiment disclosed herein. Images were captured on Day 3, Day 7, Day 15 and Day 21.

FIG. 4 shows images obtained from porcine burn wound healing studies of 10% PLGA-RGD in accordance with an embodiment disclosed herein. Images were captured on Day 3, Day 7, Day 15 and Day 21.

FIG. 5 shows images obtained from porcine burn wound healing studies of 10% PLGA-HA in accordance with an embodiment disclosed herein. Images were captured on Day 3, Day 7, Day 15 and Day 21.

FIG. 6 shows images obtained from porcine burn wound healing studies of positive control (i.e. a commercial dressing e.g., for burns or second degree burns, herein referred to as “Biobrane”). Images were captured on Day 3, Day 7, Day 15 and Day 21.

FIG. 7 shows images obtained from porcine full thickness excisional is wound healing studies of 5% PLGA-RGD, 5% PLGA-HA and positive control (i.e. a clinical dressing, herein referred to as “Allevyn™”) in accordance with various embodiments disclosed herein. Images were captured on Day 0, Day 3, Day 7, Day 10, Day 14, Day 17 and Day 21.

FIG. 8 shows wound closure rate (%) from porcine full thickness wound healing studies in accordance with various embodiments disclosed herein.

FIG. 9 to 17 show a comparison of bio-implanted murine skin tissues using bioactive poly(lactic-co-glycolic acid) (PLGA) material in accordance with various embodiments disclosed herein.

FIG. 9 shows an image obtained from immunohistochemistry (IHC) staining for CD3⁺ cells, with cell nuclei (arrows) and CD3 (circles) differentially labelled. Arrows represent cell nuclei while circles represent CD3. Pathological assessment reports 2+ for negative control. Image shown is representative with at least 4 C57BL/6 mice per group. Scale bar=50 μm.

FIG. 10 shows an image obtained from Hematoxylin and Eosin (H&E) staining, with nuclear component (hematoxylin) and cytoplasmic components (eosin) differentially stained. Pathological assessment reports 2+ for negative control. Image shown is representative with at least 4 C57BL/6 mice per group. Scale bar=50 μm.

FIG. 11 shows an image obtained from immunohistochemistry (IHC) staining for CD3⁺ cells, with cell nuclei (arrows) and CD3 (circles) differentially labelled. Arrows represent cell nuclei while circles represent CD3. Pathological assessment reports 2+ for PLGA. Image shown is representative with at least 4 C57BL/6 mice per group. Scale bar=50 μm.

FIG. 12 shows an image obtained from Hematoxylin and Eosin (H&E) staining, with nuclear component (hematoxylin) and cytoplasmic components (eosin) differentially stained. Pathological assessment reports 2+ for PLGA. Image shown is representative with at least 4 C57BL/6 mice per group. Scale bar=50 μm.

FIG. 13 shows an image obtained from immunohistochemistry (IHC) staining for CD3⁺ cells, with cell nuclei (arrows) and CD3 (circles) differentially labelled. Arrows represent cell nuclei while circles represent CD3. Pathological assessment reports 2+ for PLGA 10%. Image shown is representative with at least 4 C57BL/6 mice per group. Scale bar=50 μm.

FIG. 14 shows an image obtained from Hematoxylin and Eosin (H&E) staining, with nuclear component (hematoxylin) and cytoplasmic components (eosin) differentially stained. Pathological assessment reports 2+ for PLGA 10%. Image shown is representative with at least 4 C57BL/6 mice per group. Scale bar=50 μm.

FIG. 15 shows an image obtained from immunohistochemistry (IHC) staining for CD3⁺ cells, with cell nuclei (arrows) and CD3 (circles) differentially labelled. Arrows represent cell nuclei while circles represent CD3. Pathological assessment reports 1+ for PLGA 20%. Image shown is representative with at least 4 C57BL/6 mice per group. Scale bar=50 μm.

FIG. 16 shows an image obtained from Hematoxylin and Eosin (H&E) staining, with nuclear component (hematoxylin) and cytoplasmic components (eosin) differentially stained. Pathological assessment reports 1+ for PLGA 20%. Image shown is representative with at least 4 C57BL/6 mice per group. Scale bar=50 μm.

FIG. 17 shows an image obtained from Hematoxylin and Eosin (H&E) staining, with nuclear component (hematoxylin) and cytoplasmic components (eosin) differentially stained. Pathological assessment reports 1+ for PLGA 20%. Image shown is representative with at least 4 C57BL/6 mice per group. Scale bar=200 μm.

FIG. 18 shows native view of dermal template model designed in accordance with various embodiments disclosed herein.

FIG. 19 shows layer view of dermal template model designed in accordance with various embodiments disclosed herein.

FIG. 20 shows layout of partial thickness burn wounds on flank of pig.

FIG. 21 shows layout of full thickness excisional wounds on flank of pig.

### EXAMPLES

Example embodiments of the disclosure will be better understood and readily apparent to one of ordinary skill in the art from the following examples, tables and if applicable, in conjunction with the figures. It should be appreciated that other modifications related to structural, and/or chemical changes may be made without deviating from the scope of the invention. Example embodiments are not necessarily mutually exclusive as some may be combined with one or more embodiments to form new example embodiments. The example embodiments should not be construed as limiting the scope of the disclosure.

The following examples describe the development of 3D printed porous sheets that comprises bioactive PLGA as bioadditive for skin regeneration and biodegradable PLGA base polymer. Bioactive PLGA was blended at specific ratios in PLGA to create a formulation for 3D Printing into porous sheets. The porous sheets are 3D printable for unique porosity and product customization. Advantageously, as will be shown in the following examples, the bioactive PLGA material designed in accordance with various embodiments disclosed herein is capable of encouraging skin regeneration, reepithelization and reducing inflammation (e.g. of the skin), demonstrating its suitability for use in wound care (e.g., as dermal template in burns wound treatment). Even more advantageously, the bioactive PLGA material designed in accordance with various embodiments disclosed herein demonstrates that the aforementioned several technical advantages and effects are achievable without the use of stem cells.

In the following examples, the efficacy of the bioactive PLGA material is demonstrated on both partial thickness and full thickness wounds. Partial thickness burns wound model on porcine showed excellent healing outcomes relative to positive and negative controls. In vivo full thickness porcine excisional wound model study also showed excellent healing outcomes relative to positive and negative controls.

### Example 1: Bioactive PLGA Three-Dimensional Printed Structure

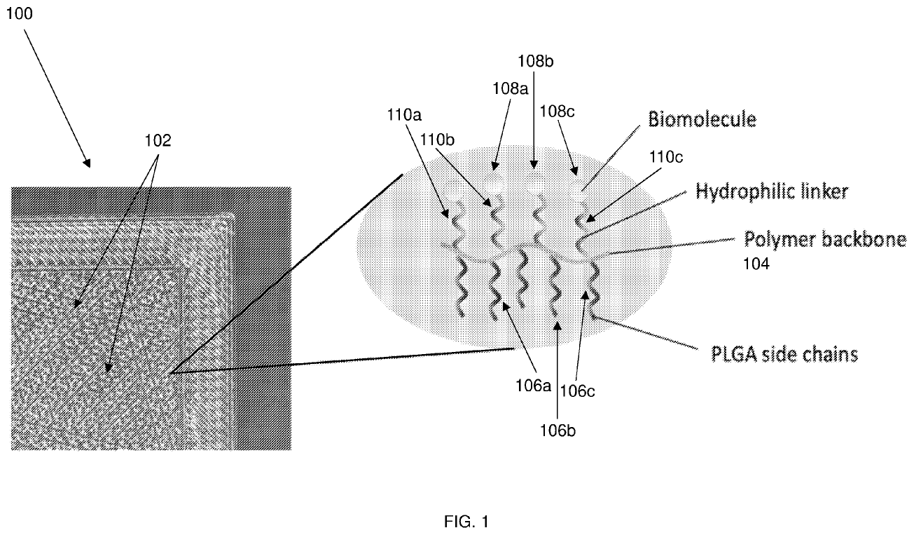

Bioactive PLGA containing RGD peptides or short chain hyaluronic acid (HA, 8-13 saccharide units) are used as bioadditives in PLGA base polymers. PLGA is a biodegradable polymer that can be used in drug delivery, bone scaffolds and other biomedical applications. The bioadditives are first blended in varying ratio (e.g., 5-20 wt % for PLGA-RGD brush copolymer and 10 wt % for PLGA-HA brush copolymer) using a mixer. Once the additives are evenly blended into PLGA, the mixture is extruded into filaments using a twin screw extruder to obtain 3DP quality filaments. Flexible sheets with micropores are created using 3DP to obtain porous scaffolds that would function as dermal templates (FIG. 1). The samples are then tested on porcine wound healing models for efficacy.

Porcine wound healing histology data has shown epidermal regeneration by Day 7 and reduced inflammation on Day 15 with 5% PLGA-RGD. The healing outcome is remarkably improved with using 5% PLGA-RGD as compared to higher blending ratio. Such results are unexpected and against conventional wisdom since one skilled in the art would expect better results using higher ratio of bioactives.

### PLGA as Choice of Material

In various embodiments, the material was chosen based on its biovalidation test performance where the design was incorporated subsequently using the selected material. It will be appreciated that as the dermal template needs to be biodegradable, only biodegradable polymers such as poly(lactic-co-glycolic acid) (PLGA), poly(lactic acid) (PLA) and poly(caprolactone) (PCL) may be used. However, through the experiments performed, the inventors found that PCL did not perform as well as PLGA in biovalidation tests, preventing its use in a dermal template. PLA has a long degradation time of at least 2 years, which is non-ideal for a dermal template. Hence, PLGA was chosen for fast degradation and flexibility of printed sheets.

### Example 2: Partial Thickness Burn Wounds

Briefly, nine 5×5 cm² partial thickness burn wounds were created on the flank of a male juvenile Yorkshire pig (4 mth), for each of 3 pigs, using a metal brand immersed in hot water bath (95° C.). The brand was pressed on the pig's skin for 5 s before removal and the wounds were cleaned with chlorhexidine and saline before samples were applied. The samples, sham and positive control of Biobrane (gold standard for burns), were randomly distributed on each of the 9 wounds for each pig, with equal representation of each sample on all 3 sections of the pig's dorsal (top, middle, bottom).

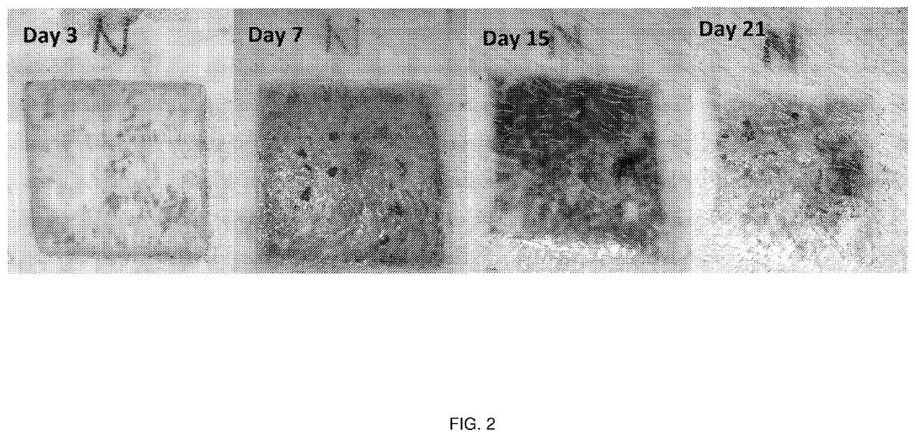

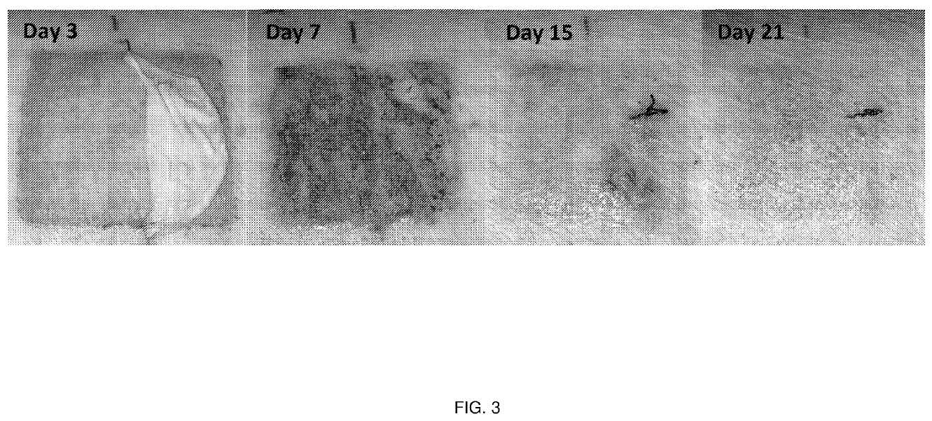

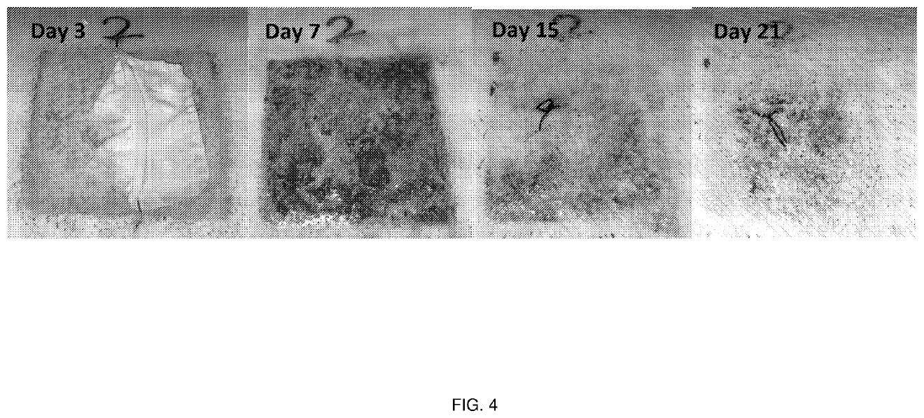

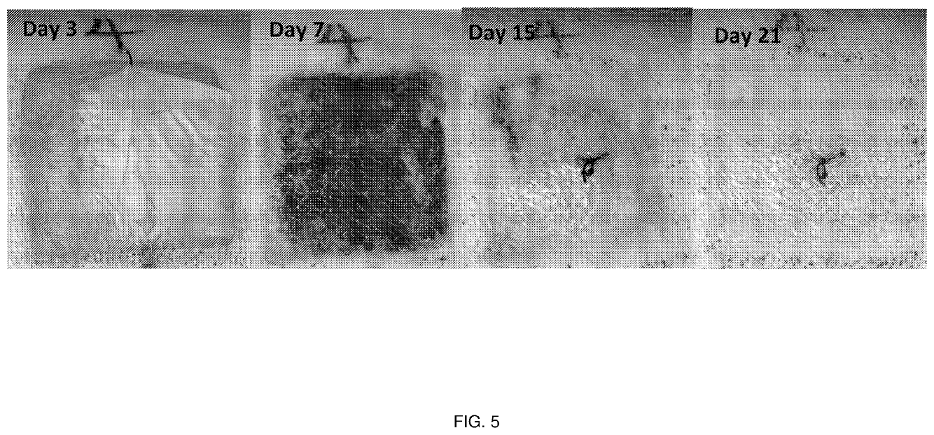

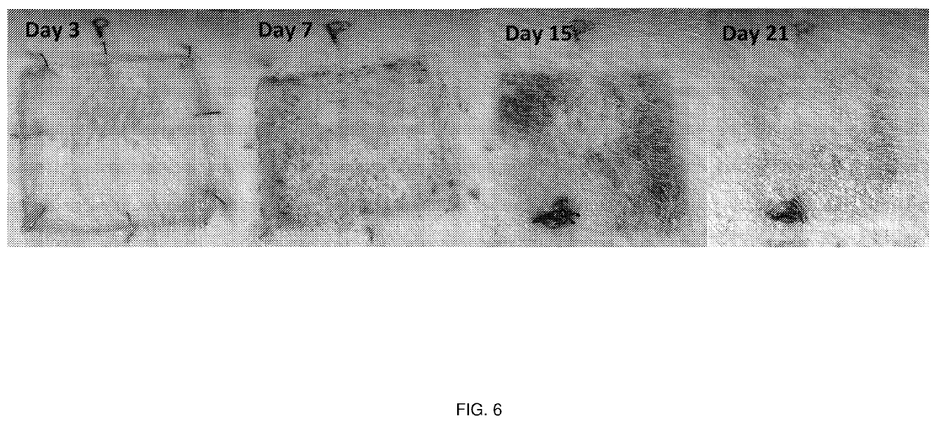

From visual assessment by a trained plastic surgeon, samples from PLGA-RGD (5% and 10% PLGA-RGD brush copolymer in PLGA) and PLGA-HA (10% PLGA-hyaluronic acid, HA 8-13 saccharide units) showed complete healing on day 21 with negligible inflammation. On day 15, wounds treated with 5% PLGA-RGD healed well in 2 of 3 pigs whereas wounds treated with 10% PLGA-HA healed well in all 3 pigs. Wounds treated with 10% PLGA-RGD, 20% PLGA-RGD and Biobrane showed good healing in only 1 out of 3 pigs. Wounds treated with 5% PLGA-RGD and 10% PLGA-HA had negligible inflammation at day 21. Some slight inflammation was still observed in the wound treated with Biobrane on day 21. Negative control of no treatment, still had slough and showed significant inflammation even at day 21 (FIG. 2, FIG. 3, FIG. 4, FIG. 5, and FIG. 6). From this preliminary in vivo burns study, it can be deduced that 3DP bioactive PLGA can indeed encourage healing in partial thickness burn wounds and the healing outcome is at least on par with gold standard, Biobrane. In view of the extremely encouraging results, further optimization of the materials may be carried out to obtain improved printing parameters, scaffold design and formulation for a dermal template to treat burn wounds. From the data, it is also evident that filament extrusion and 3DP by Fused Filament Fabrication methods do not deactivate the biomolecules present in the material. Hence, it is shown that a good method has been developed to produce 3DP dermal templates for use on burn wounds.

The bioactive PLGA samples were observed to roll over on day 3, despite much coverage with Biatain foam dressings, hypafix tape, opsite surgical drape, cotton gamgee padding and coverage with pig jacket. Aside from pig jacket, the additional dressings were meant to protect wound bed and skin scaffold from contamination or mechanical abrasion, a standard practice on burns wound treatment on human burns patients. This could be due to the 3DP samples not being secured firmly by stapling down to skin on all four sides. Nevertheless, the short contact time between scaffold and wound already allowed the bioactives in the material to encourage wound healing, as evident from the in vivo porcine studies. In view of the encouraging results, optimization of the printing for further in vivo studies may be carried out. No rolling over of positive control was observed as the plastic surgeon was certain that Biobrane needed to be stapled with 8 staples all around to secure the material on skin, from his clinical experience.

### Example 3: Full Thickness Excisional Wounds

Briefly, full thickness excisional wounds were created by removing 3×3 cm² of full thickness skin in the cephalad, middle and caudal areas of each pig. Incisions were made along the wound edges with a surgical blade to the panniculus carnosus layer and the overlying skin was excised. The wounds were cleaned with chlorhexidine and saline before samples were applied. The samples, and control of Allevyn™ by Smith & Nephew (clinical dressing for chronic wound) were randomly distributed on each of the 2 wounds for each pig with equal representation. After treatment application, samples were immobilized firmly by 4 skin staples and protective cover (gauze/Gamgee pad/Opsite™/pig jacket) were performed to protect the wound bed. Wound cleaning and samples changing were performed every 3-4 days through the entire study duration day 0-day 21. Wound closure measurement was performed on day 10, 14, 17, 21.

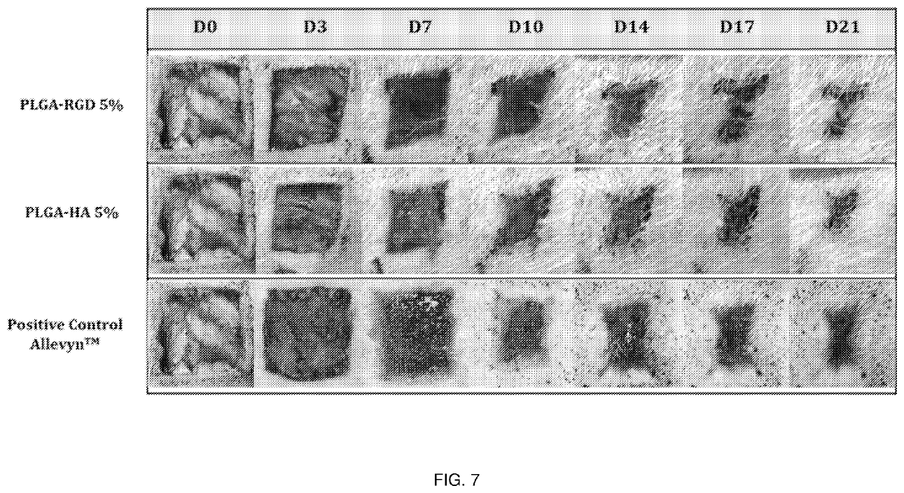

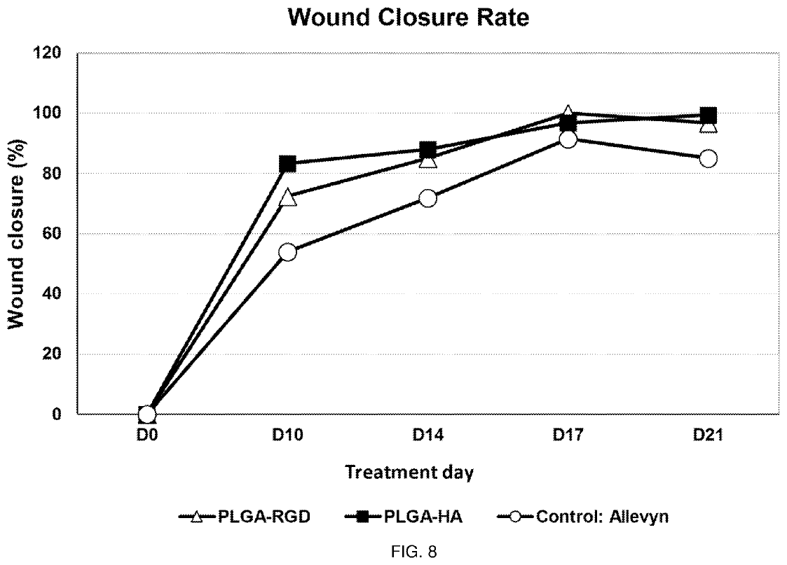

From visual assessment by a trained vascular surgeon, samples from PLGA-RGD (5% PLGA-RGD brush copolymer in PLGA) and PLGA-HA (5% PLGA-hyaluronic acid, HA 8-13 saccharide units), showed complete healing on day 21 with scab and no inflammation (FIG. 7). From the data analysis on wound closure measurement, on day 10, wounds treated by PLGA-RGD and PLGA-HA healed well with >70% re-epithelization, which is more than 10%-20% acceleration in healing speed than that of Allevyn™ (<60% re-epithelization on day 10) (FIG. 8). From this preliminary in vivo full thickness wound model study, it can be deduced that 3DP bioactive PLGA can indeed encourage wound healing in full thickness excisional wounds and the healing outcome is at least on par with clinical standard, Allevyn™. In view of the extremely encouraging results, further optimization of the materials may be carried out to obtain improved printing parameters, scaffold design and formulation for a dermal template to treat excisional wounds. From the data, it is also evident that filament extrusion and 3DP by Fused Filament Fabrication methods do not deactivate the biomolecules present in the material. Hence, it has been shown that a good method has been developed to produce 3DP dermal templates for use on full thickness excisional wounds.

### Example 4: Inflammatory Response

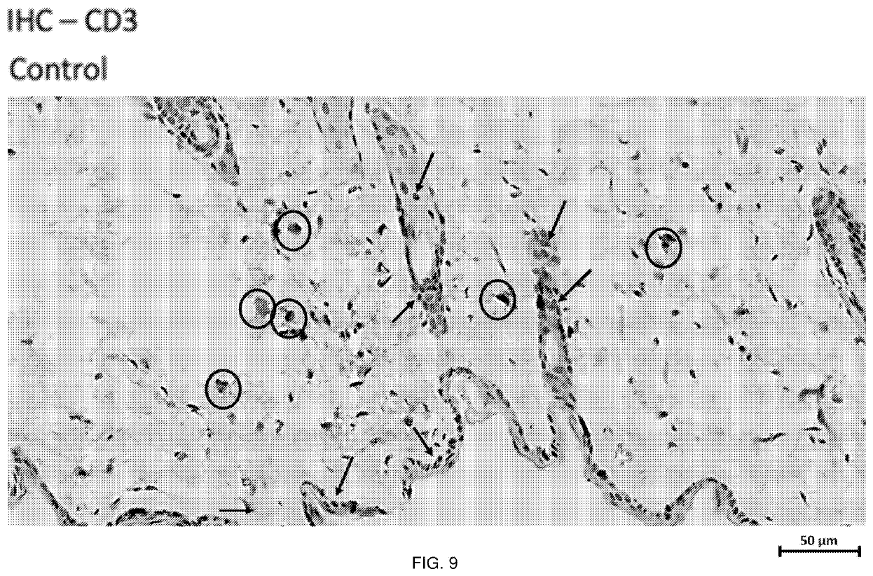

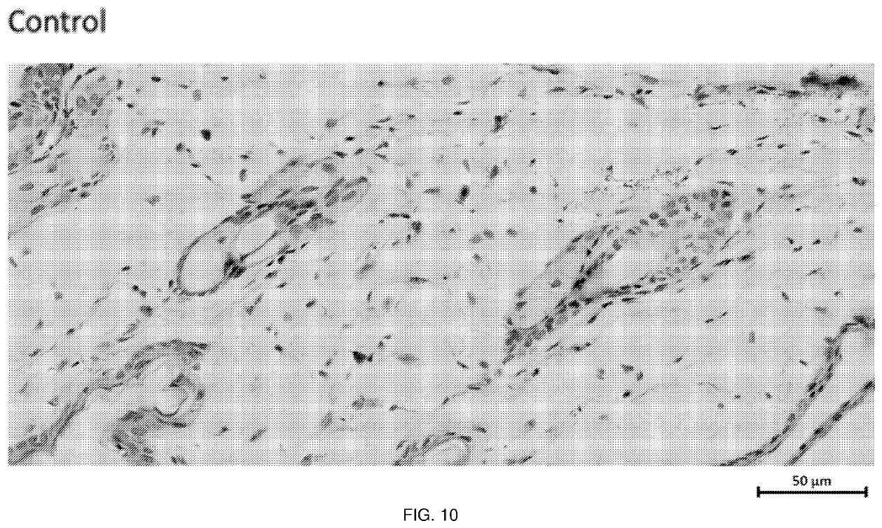

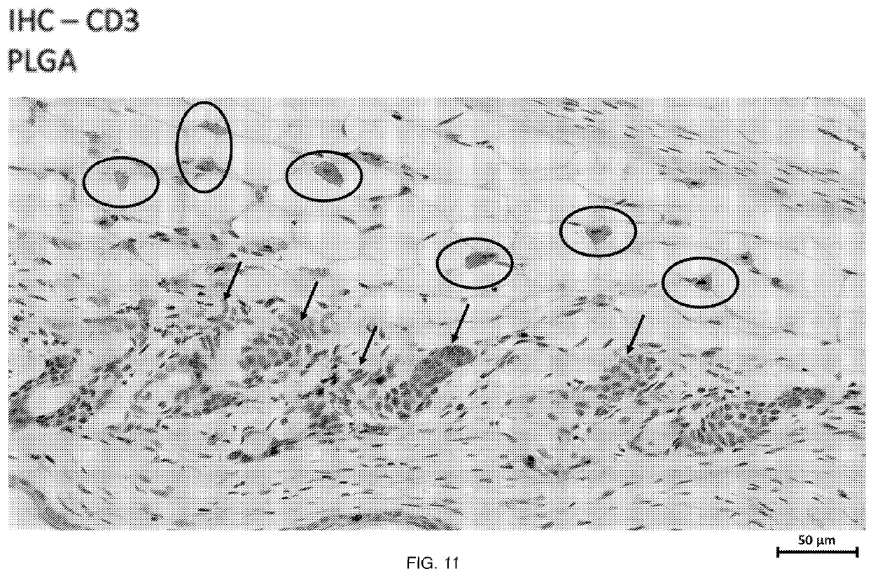

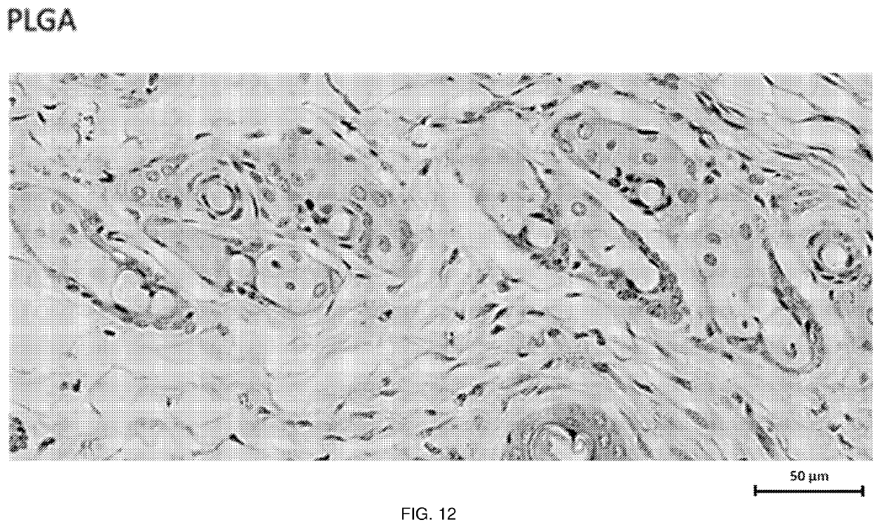

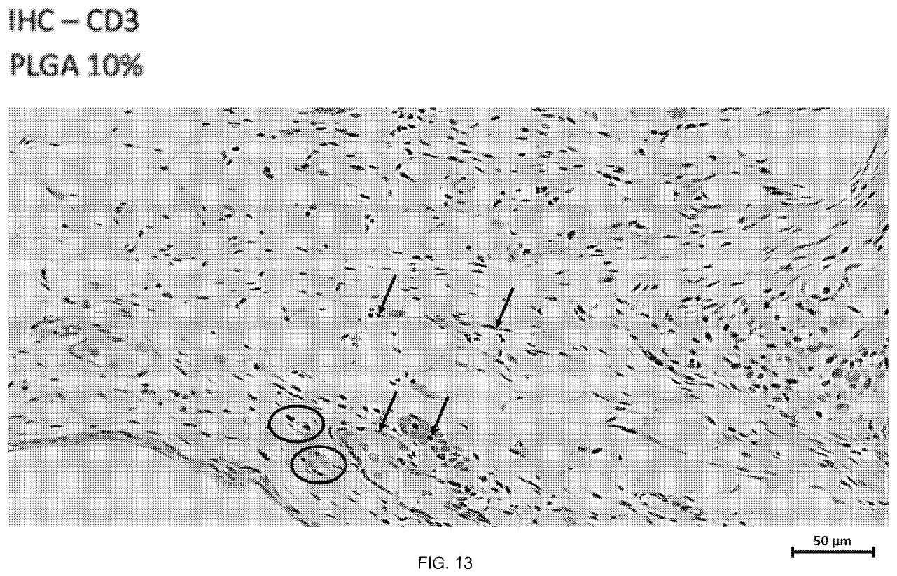

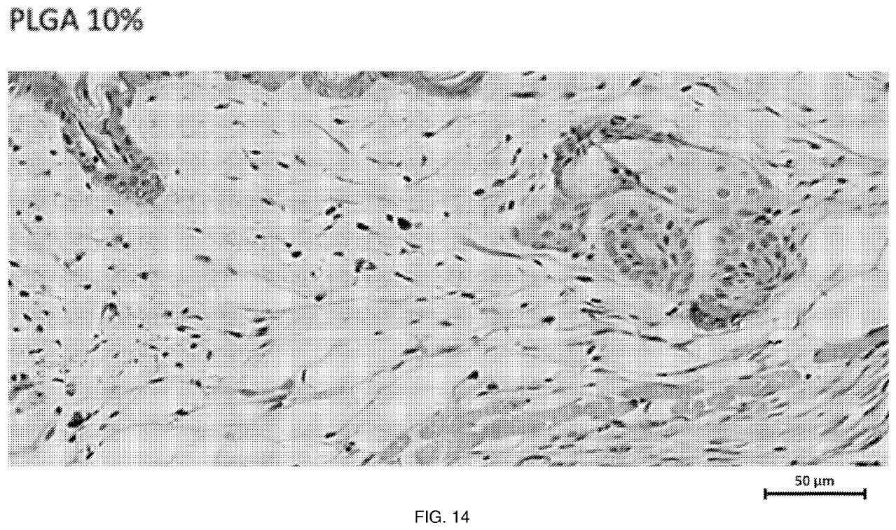

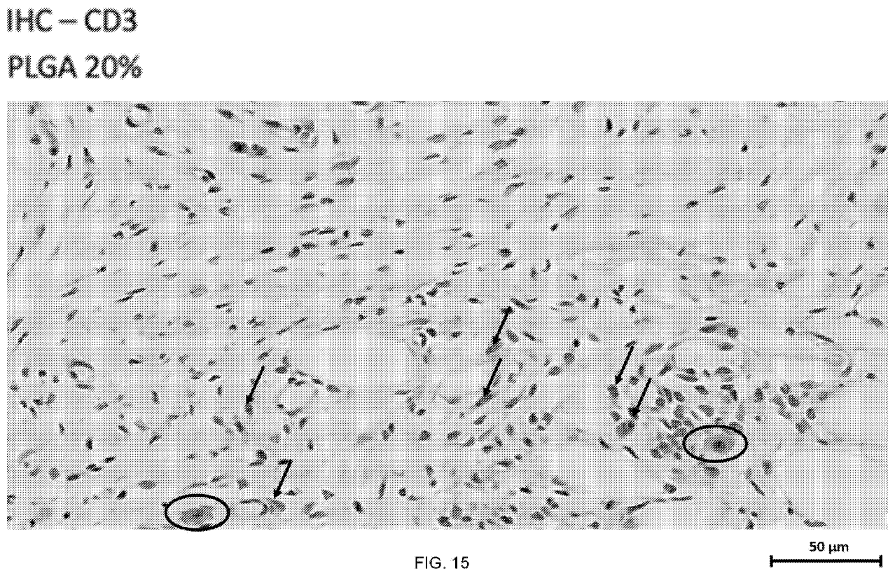

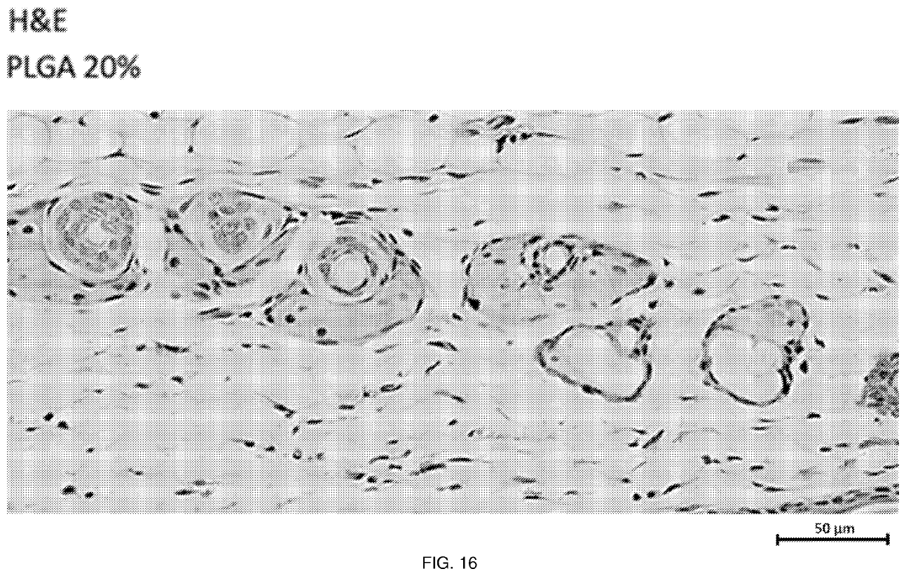

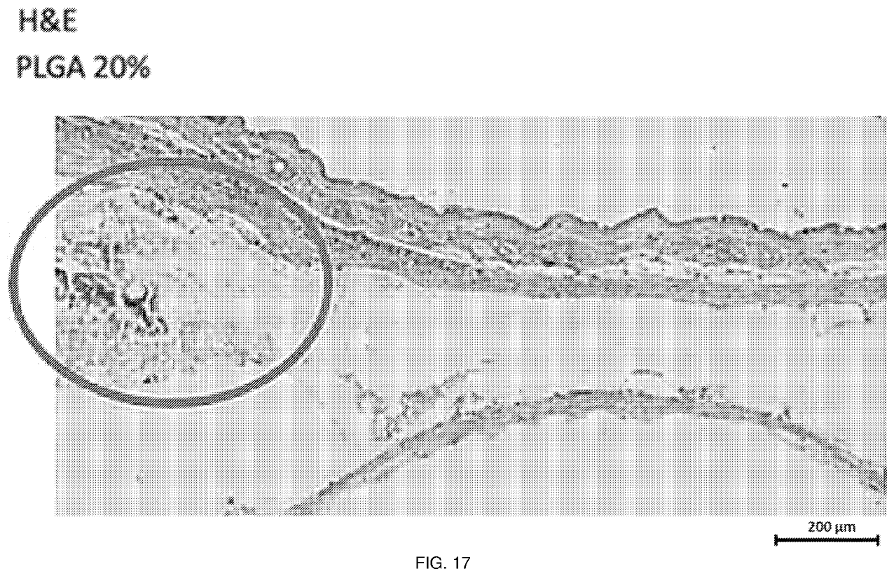

Inflammatory response tests are also conducted on the materials using mouse models to determine whether the materials elicit inflammation in vivo. A 28-day study using 8 weeks old female C57BL/6 wild type mice with material samples randomly assigned to mouse groups, with 4 mice per sample group, was carried out twice, using 2 different batches of materials. It was found that the PLGA-RGD samples either do not elicit additional inflammatory response in mice (10% PLGA-RGD) or cause a reduction in inflammation (20% PLGA-RGD), compared to sham (no treatment) or pure PLGA sample groups (FIG. 9 to FIG. 17). Pathological assessment showed reduction in inflammation in 3DP samples with 20% PLGA-RGD compared to negative control (no implant) and positive control of pure PLGA. All images shown are representative with at least 4 C57BL/6 mice per group. H&E stained image of murine tissues in FIG. 17 shows wound space being partially filled by connective tissues while PLGA scaffold bioresorbs (circled).

Based on both the murine biocompatibility studies and porcine wound healing studies, it is concluded that the 3DP bioactive PLGA is capable of enhancing wound healing in burn/excisional wounds with reduced inflammation, as compared to both positive and negative controls.

### Example 5: Experimental

Bioactive PLGA material synthesis is described in PCT/SG2020/050621, the contents of which are fully incorporated herein.

Porcine wound healing model studies are carried out under IACUC study 2022/SHS/1702. Animals are handled by a trained vet and a plastic surgeon, Dr James Mok. Three 4 month old male Yorkshire pigs of average weight of 32 kg were used in this study. The studies were carried out in National Large Animal Research Facility (NLARF), Singapore.

Porcine full thickness excisional wound healing model studies are carried out under IACUC study 2022/SHS/1732. Animals are handled by a trained vet, a vascular surgeon and a dermatologist, Dr Joseph Lo and Dr Tan Chee Hian.

Three 3-month-old male Yorkshire pigs of average weight of 32 kg were used in this study. The studies were carried out in National Large Animal Research Facility (NLARF), Singapore.

### Example 6: Design of Dermal Template and 3D Printing Parameters

Dermal template model (56×56×0.12 mm) was created using Ultimaker Cura software in addition to the optimisation of 3D printing parameters. Subsequently, the software converts the model into a readable format (gcode) for the 3D printer. The dermal template was fabricated by fused filament fabrication method (FFF) of 3D printing using Ultimaker S5 dual nozzle 3D printer. Micropores on the dermal template were designed to allow moisture to be extracted away from the wound but retains sufficient material to promote wound healing. In addition to these, the micropores must not affect the flexibility of the skin scaffold as it needs to satisfactorily conform to the wound site & its surrounding areas.

### Design of Micropores

The design of micropores utilizes functions in Ultimaker Cura software through varying infill density and available infill patterns which include grid, lines, triangles and many more. However, in some embodiments, the selection of infill patterns is limited to grid and triangles only due to the desired thickness of the dermal template and pattern shape. Through the experiments performed, the inventors found that patterns other than grid and triangles may produce wider line distance at the same infill density which gives rise to higher porosity. As the desired thickness ranges between 0.12 to 0.2 mm, printing process becomes difficult as the part/template becomes thinner (which requires the nozzle to be positioned at 0.06 to 0.1 mm away from its printing layer, which is also referred to as the layer height). At such a small layer height with a default material flow rate, the molten material exiting the nozzle will tend to be “squished”, thereby creating thicker line widths than the set value which alters the actual porosity or infill density. In an example, 2 infill patterns, namely grid & triangles, were selected and experimented for the design of the micropores and triangle pattern was found to be the most consistent pattern in terms of actual infill density and print quality with smaller micropores at an infill density between 90 to 95%.

Table 1 below shows the 3D printing parameters used for dermal template fabrication.

TABLE 1
3D printing parameters of dermal template

| Parameters | Value | |
|---|---|---|
| Print Temperature | 130-140° | C. |
| Core Type/Size | AA/0.25 | mm |
| Buildplate Temperature | 45-60° | C. |
| Buildplate Adhesion Type | Skirt | |
| Infill Density | 100% | |
| Infill Pattern, Orientation | Lines, ±45° | |
| Infill Overlap | 0.1 | mm |
| Print Speed | 20-55 | mm/s |
| Layer Height | 0.06 | mm |
| Initial Layer Horizontal Expansion | 0 | mm |
| No. of Top Layer | 0 | |
| No. of Bottom Layer | 0 | |
| Line Width | 0.23-0.25 | mm |
| Fan Speed | 30-100% | |
| Wall Line Count | 2 | |
| Buildplate Surface | Glass | |
| Support Blocker (SB) | Enabled | |
| SB Dimensions | 50 × 50 × 5 | mm |

| | |
|---|---|
| SB Wall Line Count | 2 |
| SB Infill Density | 90-95% |
| SB Infill Pattern | Triangles |

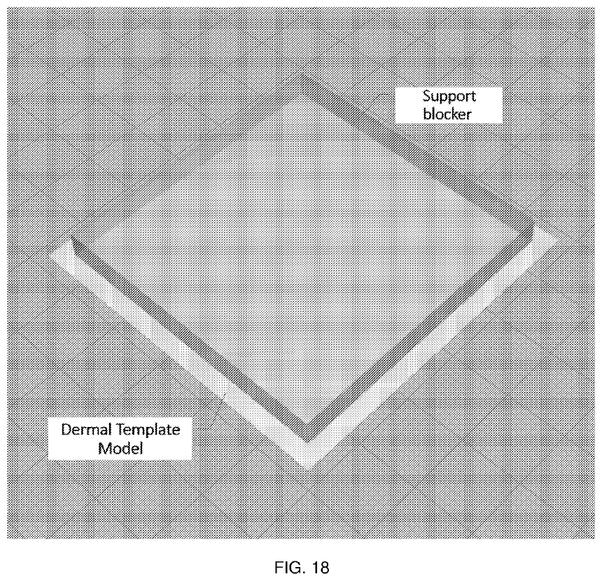

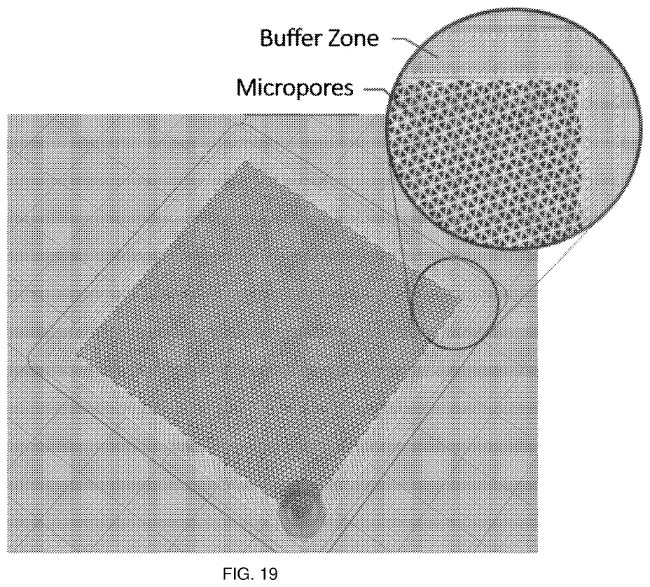

The design of dermal template incorporated a buffer zone surrounding the micropores to allow ease of application while reducing damage risk to the dermal template during and after application. This was achieved by using the support blocker function in Ultimaker Cura where it allowed dedicated control over the infill pattern and density of the micropores without affecting the buffer zone. FIG. 18 and FIG. 19 show the dermal template model in native and layer views respectively.

### Design of Printed Material

In various embodiments, the design principle focuses on retaining the most amount of material within the 3D printed part volume to maximize the wound healing effect imparted by the copolymers while micropores were considered for both moisture extraction and oxygen penetration, which are parameters essential for wound healing. Therefore, micropores were designed to be as small as possible, within the limitations of the FFF 3D printer, where it is sufficient for moisture to be extracted away from the wound but does not remove excessive amount of materials interacting with the wound.

In various embodiments, the micropores are designed to allow wound fluid transfer from wound to secondary dressing used on top of the skin substitute. This is an important property for a good, effective skin substitute. Without the pores, wound fluid may be trapped within the wound, keeping it wet and delaying healing. Wet wounds are also prone to infections which can lead to sepsis and non-healing wounds.

### Example 7: Experimental Design of Porcine Burn Wound Model

Number of wounds created on each animal: 9 wounds of 5×5 cm² each.

Treatment of pig before wounding—Animal was on anaesthetic and given preprocedural antibiotics with monitoring of vitals. Locations on the pig where wounds are to be created, are outlined with an alcohol based marker, and shaved off hair with a razor before decontaminating with iodine solution. Each of these outlined wound locations are photographed. The superficial partial thickness burn wounds are created on the pig dorsum. The underlying muscles are inferior trapezius, latissimus dorsi, external abdominal oblique. The burn wounds are not expected to reach muscular depth, nor will it impede muscular movement. They are created unilaterally.

Method of burns wound creation—a solid anodized aluminium block with 5×5 cm² face, is immersed in hot water bath with fixed temperature for 1 min. The block is lifted out and pressed onto demarcated wound locations on the pig for a duration of 5 seconds to produce a partial thickness burn wound 5×5 cm in size before block is returned to hot water bath. A handheld timer was used to regulate the contact duration. Each area was separated from other wound areas by at least 3 cm to prevent site-to-site influence.

Preparation of the wound bed—Each wound that is properly scalded will have the epidermis beginning to slough off. Sterile gauze soaked in sterile saline is used to scrub each of the wounds to remove dead skin to optimize the wound bed for dressing as per treatment arm. The wound is debrided until pink healthy tissue is seen, with minimal white tissue left (dead tissue). The burn wounds are allowed to cool, tattooing of the burn margins are performed with methylene blue and 21G needle and photos are taken.

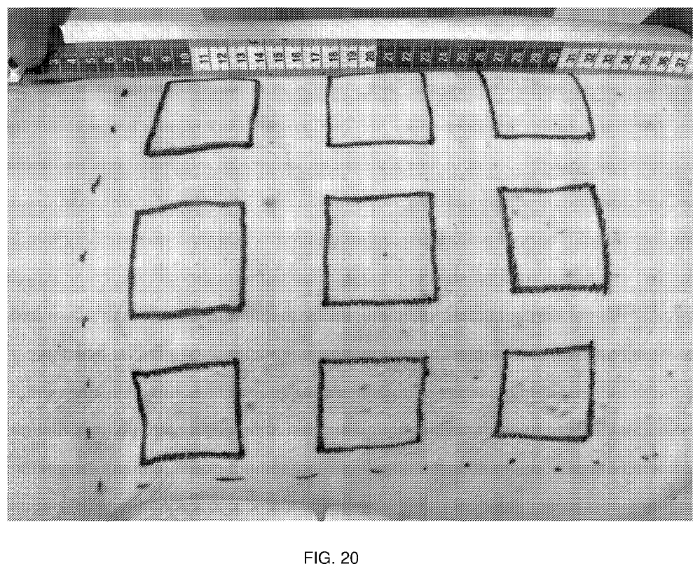

Application of the burns dressing—Randomised allocation to treatment groups will determine type of primary dressing applied to sequential wound locations along the pig flank (FIG. 20). Sterile dressing is applied to the wound and secured with skin staples. Foam dressing (Biatain) overlay is placed over all wounds for mechanical protection and to absorb excess exudate. This is similar to current gold standard treatment for humans. A transparent adhesive polyurethane film (e.g. Opsite) is applied to the outer layers as a protective covering. Cotton pads are used as a cushion before bandage is applied and taped down with Elastoplast. A pig jacket is then put over the animal. This is to reduce the risk of the animal rubbing itself against edges etc that would dislodge the dressing and affect the experimental outcome.

7.1. Wound healing

Under anaesthesia, the outer covering of Biatain/Opsite/cottonpads/bandage was removed and the wounds inspected on Day 3, 7, 15, 21 and photographed to monitor wound healing progress. The wounds were dressed back in same fashion using new dressings. Skin biopsy (0.5×0.5 cm) was taken at Day 7 and Day 15. The biopsy wound was sutured with silk 2/0 and dressed. At 21 days post-treatment, the study was terminated and the animals euthanized.

7.2. In vivo biocompatibility study using murine model

Eight weeks old female C57BL/6 wild type mice were purchased from InVivos Pte Ltd and randomly assigned to one of the following four groups: (1) Control, (2) PLGA, (3) PLGA-RGD 10% and (4) PLGA-RGD 20%. All groups had at least 4 mice. 3DP coupons for in vivo assays were dimensioned to 5×5×0.4 mm. Briefly, each mice was anaesthetised with ketamine/xylazine and a small incision was made on the upper dorsal back. Upon making an incision, scissors was used to separate the skin and subcutaneous tissue in order to create a pocket for the coupon insertion. One coupon was then inserted into the subcutaneous space under the skin of each mice. For mice in the control group, an incision was made and a pocket was formed. No coupon was inserted and the mice were sutured up as per the other experimental groups. Four weeks post implantation, all mice were sacrificed. Mouse skin surrounding the implant was harvested, fixed in formalin and embedded in paraffin for histological studies. Haematoxylin and Eosin (H&E) staining, as well as immunohistochemistry staining for CD3 (clone SP162) were performed on these skin sections. CD3 was selected as a marker as it is a T cell lineage marker and could be used to detect the presence and level of inflammation around the site of implantation. These tissue sections were stained with standard immunohistochemistry DAB protocol, using hematoxylin as counterstain. Appropriate controls were included and images were captured using Zeiss Axio Scan Z1 slide scanner. All images shown are representative with at least 4 C57BL/6 mice per group. Trained pathologist scored the H&E and CD3 IHC stained images blinded to the conditions in a semi-quantitative method that categorized the amount of immune infiltrates into 4 different categories, ranging from no/low infiltrates to abundant infiltrates. The scores were described as “0+”, “1+”, “2+” and “3+”. All animal handling procedures were approved by the Institutional Animal Care and Use Committee and conformed to the National Advisory Committee for Laboratory Animal Research Guidelines (IACUC #201550).

### Example 8: Experimental Design of Porcine Full Thickness Excisional Wound Model

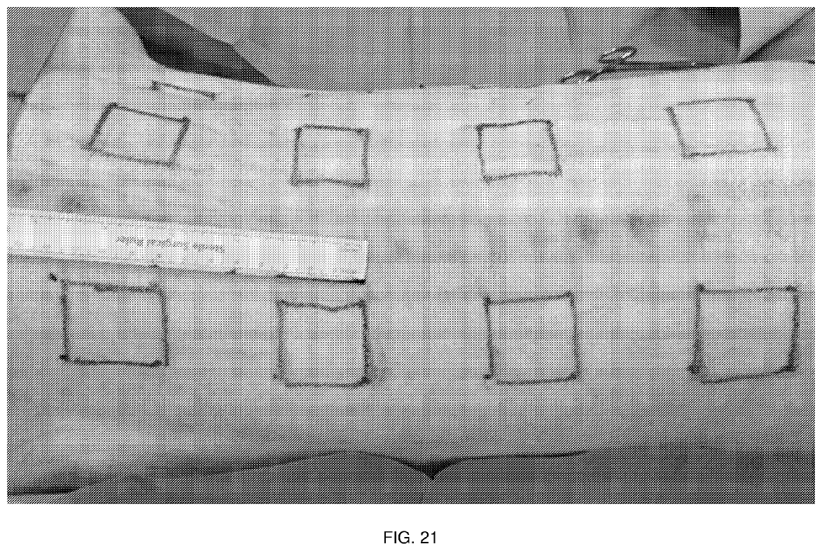

Treatment of pig before wounding—The animals were anesthetized. Locations on the pig where wounds are to be created, are outlined with an alcohol based marker, and shaved off hair with a razor before decontaminating with iodine solution. Each of these outlined wound locations are photographed. The full thickness excisional wounds are created on the pig dorsum (FIG. 21).

Method of full thickness excisional wound creation—areas of skin wound were created by removing 3 cm×3 cm of full thickness skin in the central back along the cephalad, middle and caudal areas. Incisions were made along the wound edges with a surgical blade to the panniculus carnosus layer and the overlying skin was excised. Each area was separated from other wound areas by at least 5 cm to prevent site-to-site influence.

Preparation of the wound bed—Sterile gauze soaked in sterile saline is used to scrub each of the wounds to optimize the wound bed for dressing as per treatment arm. Photos are taken.

Application of samples—Randomised allocation to treatment groups will determine type of primary dressing applied to sequential wound locations along the pig flank. Samples were secured with skin staples. Gauze and gamgee pad are placed over all wounds for mechanical protection and to absorb excess exudate. This is similar to current gold standard treatment for humans. A transparent adhesive polyurethane film (e.g. Opsite™) is applied to the outer layers as a protective covering. A pig jacket is then put over the animal. This is to reduce the risk of the animal rubbing itself against edges etc that would dislodge the dressing and affect the experimental outcome.

### 8.1. Wound Healing

Under anaesthesia, the outer covering of Gauze/Gamgee pad/Opsite™/bandage was removed and the wounds inspected on Day 0, 3, 7, 10, 14, 17, 21 and photographed to monitor wound healing progress. The wounds were dressed back in same fashion using new dressings. Skin biopsy (0.5×0.5 cm) was taken at Day 7 and Day 14. The biopsy wound was sutured with silk 2/0 and dressed. At 21 days post-treatment, the study was terminated and the animals euthanized.

8.2. In vivo biocompatibility study using murine model

Eight weeks old female C57BL/6 wild type mice were purchased from InVivos Pte Ltd and randomly assigned to one of the following four groups: (1) Control, (2) PLGA, (3) PLGA-RGD 10% and (4) PLGA-RGD 20%. All groups had at least 4 mice. 3DP coupons for in vivo assays were dimensioned to 5×5×0.4 mm. Briefly, each mice was anaesthetised with ketamine/xylazine and a small incision was made on the upper dorsal back. Upon making an incision, scissors was used to separate the skin and subcutaneous tissue in order to create a pocket for the coupon insertion. One coupon was then inserted into the subcutaneous space under the skin of each mice. For mice in the control group, an incision was made and a pocket was formed. No coupon was inserted and the mice were sutured up as per the other experimental groups. Four weeks post implantation, all mice were sacrificed. Mouse skin surrounding the implant was harvested, fixed in formalin and embedded in paraffin for histological studies. Haematoxylin and Eosin (H&E) staining, as well as immunohistochemistry staining for CD3 (clone SP162) were performed on these skin sections. CD3 was selected as a marker as it is a T cell lineage marker and could be used to detect the presence and level of inflammation around the site of implantation. These tissue sections were stained with standard immunohistochemistry DAB protocol, using hematoxylin as counterstain. Appropriate controls were included and images were captured using Zeiss Axio Scan Z1 slide scanner. All images shown are representative with at least 4 C57BL/6 mice per group. Trained pathologist scored the H&E and CD3 IHC stained images blinded to the conditions in a semi-quantitative method that categorized the amount of immune infiltrates into 4 different categories, ranging from no/low infiltrates to abundant infiltrates. The scores were described as “0+”, “1+”, “2+” and “3+”. All animal handling procedures were approved by the Institutional Animal Care and Use Committee and conformed to the National Advisory Committee for Laboratory Animal Research Guidelines (IACUC #201550).

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A regenerative material for treatment of wounds, the material comprising:
(i) a base poly(lactic-co-glycolic acid) (PLGA);
(ii) a bioactive poly(lactic-co-glycolic acid) (PLGA) copolymer; and
(iii) pores patterned on said material.

2. The regenerative material of claim 1, wherein each pore comprises a polygonal shape.

3. The regenerative material of claim 1, wherein each pore has a minimum circumscribed circle diameter of at least 100 μm.

4. The regenerative material of claim 1, wherein the distance between the pores is from 100 μm to 300 μm.

5. The regenerative material of claim 1, wherein the material comprises a buffer zone surrounding the pores.

6. The regenerative material of claim 1, wherein the material has an infill density of from 70% to 100%.

7. The regenerative material of claim 1, wherein the ratio of the (i) base PLGA to (ii) bioactive PLGA copolymer present in the material is from about 60.0-99.9:0.1-40.0.

8. The regenerative material of claim 1, wherein the material comprises from 60 wt % to 99.9 wt % of the base PLGA.

9. The regenerative material of claim 1, wherein the material comprises from 0.1 wt % to 40 wt % of the bioactive PLGA copolymer.

10. The regenerative material of claim 1, wherein the material comprises a three-dimensional (3D) printed structure or part.

11. The regenerative material of claim 1, wherein the bioactive PLGA copolymer comprises a bioactive PLGA copolymer with a poly(norbornene-dicarboximide) backbone having one or more repeating units represented by general formula (I) and one or more repeating units represented by general formula (II): 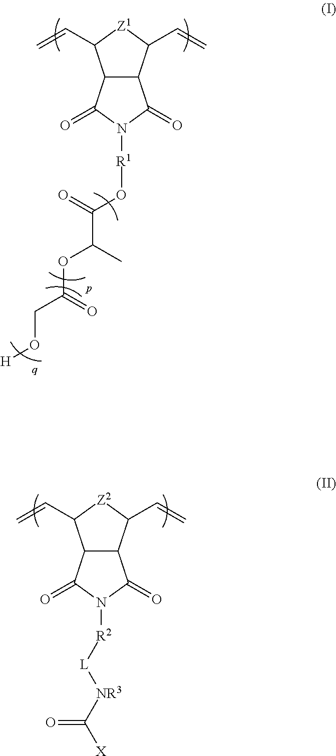
wherein
R¹ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R² is optionally substituted alkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, oligopeptides, carbohydrates, oligosaccharides, sugar, collagen, hyaluronic acid, therapeutic/drug molecules and derivatives thereof;
Z¹ and Z² are each independently selected from CR^{a}R^{b}, O, NR^{c}, SiR^{a}R^{b}, PR^{a} or S, wherein R^{a}, R^{b} and R^{c} are each independently selected from the group consisting of H, optionally substituted alkyl, optionally substituted alkenyl and optionally substituted alkynyl;
$$p \geq 1 ; and$$
$$q \geq 1.$$

12. The regenerative material of claim 1, wherein X is selected from the group consisting of RGD, SRGDS, RGDS, A5G81 (AGQWHRVSVRWGC), SVVYGLR, (IRIK)₂, (IKKI)₃, heparin oligosaccharide DP8, DP10, DP12, DP14, DP16, DGEA, (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence, hyaluronic acid and combinations thereof.

13. The regenerative material of claim 1, wherein the regenerative material is obtained by the method comprising:
(i) producing a bioactive PLGA filament; and
(ii) printing a bioactive PLGA material from the bioactive PLGA filament according to a design model to obtain a printed structure or part with pores patterned thereon.

14. The regenerative material according to claim 13, wherein the step (i) of producing a bioactive PLGA filament comprises:
(i-a) providing a base PLGA powder and a bioactive PLGA copolymer;
(i-b) mixing the base PLGA powder with the bioactive PLGA copolymer to obtain a bioactive PLGA formulation; and
(i-c) extruding a bioactive PLGA filament from the formulation.

15. The regenerative material according to claim 13, wherein the step (ii) of printing a bioactive PLGA material from the bioactive PLGA filament comprises:
(ii-a) feeding the bioactive PLGA filament into a printing apparatus;
(ii-b) applying heat to the bioactive PLGA filament to obtain a molten form of the bioactive PLGA; and
(ii-c) depositing the molten bioactive PLGA on a print bed to form a printed part or structure.

16. The regenerative material of claim 1 for use in medicine.

17.-22. (canceled)

23. A method of stimulating skin and/or tissue regeneration in a subject in need thereof,
optionally wherein the method comprises a method of treating a wound,
optionally wherein the method comprises a method of controlling and/or reducing inflammation in a subject in need thereof,
wherein the method comprising applying the regenerative material of claim 1 to a body, a wound, and/or a body part of the subject in need thereof.

24.-25. (canceled)

26. The method of claim 23, wherein the wound is selected from the group consisting of acute wounds, burns, incisions, excisions, superficial wounds, partial thickness wounds, full thickness wounds, chronic wounds, slow healing wounds, pressure sores, venous ulcers, diabetic ulcers, foot ulcers, bed sores and combinations thereof.

27. A medical device comprising the regenerative material of claim 1.

28. The medical device according to claim 27, wherein the medical device is selected from the group consisting of dermal template, dermal regeneration template, skin scaffold, wound care product, wound dressing, personal care product, beauty product and combinations thereof.
